(19) United States
(12) Patent Application Publication
(10) Pub. No.: US 2018/0000885 A1
(43) Pub. Date: Jan. 4, 2018

(54) Therapeutic Nanoparticles With High Molecular Weight Copolymers

(71) Applicant: Pfizer Inc., New York, NY (US)

(72) Inventors: David Dewitt, Cambridge, MA (US); Maria Conceicao Figueiredo, Somerville, MA (US); Hong Wang, Newton, MA (US); Gregory Troiano, Pembroke, MA (US); Young-Ho Song, Natick, MA (US)

(73) Assignee: Pfizer Inc., New York, NY (US)

(21) Appl. No.: 15/623,533

(22) Filed: Jun. 15, 2017

Related U.S. Application Data
(63) Continuation of application No. 13/880,853, filed on Nov. 7, 2014, now abandoned.
(60) Provisional application No. 61/490,778, filed on May 27, 2011.
(60) Provisional application No. 61/405,778, filed on Oct. 22, 2010.

(51) Int. Cl. A61K 38/05; A61K 31/635; A61K 31/555; A61K 31/4375; A61K 31/4184; A61K 31/365; A61K 47/59; A61K 31/337; A61K 31/196; A61K 9/51; A61K 9/00; A61K 47/60; A61K 33/24; A61K 31/407

(52) U.S. Cl. CPC A61K 38/05; A61K 33/24; A61K 31/635; A61K 31/555; A61K 31/4375; A61K 31/4184; A61K 31/407; A61K 47/593; A61K 31/337; A61K 31/196; A61K 9/5153; A61K 9/0019; A61K 47/60; A61K 31/365

(57) ABSTRACT

The present disclosure generally relates to therapeutic nanoparticles. Exemplary nanoparticles disclosed herein may include about 0.1 to about 40 weight percent of a therapeutic agent and about 10 to about 90 weight percent a diblock poly(lactic) acid-poly(ethylene)glycol copolymer or a diblock poly(lactic)-co-poly(glycolic) acid-poly(ethylene)glycol copolymer, wherein the diblock poly(lactic) acid-poly(ethylene)glycol copolymer comprises poly(lactic) acid having a number average molecule weight of about 30 kDa to about 90 kDa or the diblock poly(lactic)-co-poly(glycolic) acid-poly(ethylene)glycol copolymer comprises poly(lactic)-co-poly(glycolic) acid having a number average molecule weight of about 30 kDa to about 90 kDa.

### CROSS REFERENCE TO RELATED APPLICATIONS

This application claims priority to U.S. Provisional Application No. 61/405,778, filed Oct. 22, 2010, and U.S. Provisional Application No. 61/490,778, filed May 27, 2011, both of which are hereby incorporated by reference in their entirety.

### BACKGROUND

Systems that deliver certain drugs to a patient (e.g., targeted to a particular tissue or cell type or targeted to a specific diseased tissue but not normal tissue), or that control release of drugs have long been recognized as beneficial. For example, therapeutics that include an active drug and that are capable of locating in a particular tissue or cell type, e.g., a specific diseased tissue, may reduce the amount of the drug in body tissues that do not require treatment. This is particularly important when treating a condition such as cancer where it is desirable that a cytotoxic dose of the drug be delivered to cancer cells without killing the surrounding non-cancerous tissues. Further, such therapeutics may reduce the undesirable and sometimes life-threatening side effects common in anticancer therapy. For example, nanoparticle therapeutics may, due to their small size, evade recognition within the body allowing for targeted and controlled delivery while, e.g., remaining stable for an effective amount of time.

Therapeutics that offer such therapy and/or controlled release and/or targeted therapy must also be able to deliver an effective amount of the drug. It can be a challenge to prepare nanoparticle systems that have an appropriate amount of the drug associated with each nanoparticle, while keeping the size of the nanoparticles small enough to have advantageous delivery properties. For example, while it is desirable to load a nanoparticle with a high quantity of a therapeutic agent, nanoparticle preparations that use a drug load that is too high will result in nanoparticles that are too large for practical therapeutic usage. Further, it may be desirable for therapeutic nanoparticles to remain stable so as to, e.g., substantially limit rapid or immediate release of the therapeutic agent.

Accordingly, a need exists for new nanoparticle formulations and methods of making such nanoparticles and compositions, that can deliver therapeutic levels of drugs to treat diseases such as cancer, while also reducing patient side effects.

### SUMMARY

In one aspect, the invention provides a therapeutic nanoparticle that includes a therapeutic agent, e.g. a taxane, and a diblock poly(lactic) acid-poly(ethylene)glycol copolymer (PLA-PEG) or a diblock poly(lactic)-co-poly(glycolic) acid-poly(ethylene)glycol copolymer (PLGA-PEG), wherein the diblock poly(lactic) acid-poly(ethylene)glycol copolymer comprises poly(lactic) acid having a number average molecule weight of about 30 kDa to about 90 kDa or the diblock poly(lactic)-co-poly(glycolic) acid-poly(ethylene)glycol copolymer comprises poly(lactic)-co-poly(glycolic) acid having a number average molecule weight of about 30 kDa to about 90 kDa. In another embodiment, the invention provides a therapeutic nanoparticle that includes a therapeutic agent, e.g. a taxane, and a diblock poly(lactic) acid-poly(ethylene)glycol copolymer or a diblock poly(lactic)-co-poly(glycolic) acid-poly(ethylene)glycol copolymer, wherein the diblock poly(lactic) acid-poly(ethylene)glycol copolymer comprises a block of poly(lactic) acid having a number average molecule weight of about 40 kDa to about 90 kDa (e.g. about 45 kDa, 47 kDa, 70 kDa, 60 kDa, or 30 kDa) or the diblock poly(lactic)-co-poly(glycolic) acid-poly(ethylene)glycol copolymer comprises a block of poly(lactic)-co-poly(glycolic) acid having a number average molecule weight of about 40 kDa to about 90 kDa.

For example, disclosed herein is a therapeutic nanoparticle comprising about 0.1 to about 40 weight percent of a therapeutic agent and about 10 to about 95, or about 10 to about 90 weight percent a diblock poly(lactic) acid-poly(ethylene)glycol copolymer or a diblock poly(lactic)-co-poly(glycolic) acid-poly(ethylene)glycol copolymer. In an embodiment, the said diblock poly(lactic) acid-poly(ethylene)glycol copolymer comprises poly(lactic) acid having a number average molecule weight of about 30 kDa to about 90 kDa or about 40 kDa to about 90 kDa. In another embodiment, the diblock poly(lactic)-co-poly(glycolic) acid-poly(ethylene)glycol copolymer comprises a block of poly(lactic)-co-poly(glycolic) acid having a number average molecule weight of about 30 kDa to about 90 kDa or about 40 kDa to about 90 kDa. In an embodiment, the block of poly(lactic) acid or the poly(lactic)-co-poly(glycolic) acid has a number average molecule weight of about 50 kDa to about 80 kDa or. In another embodiment, the poly(lactic) acid or the poly(lactic)-co-poly(glycolic) acid has a number average molecule weight of about 50 kDa. In yet another embodiment, the poly(lactic) acid or the poly(lactic)-co-poly(glycolic) acid has a number average molecule weight of about 30 kDa. In a further embodiment, the diblock poly(lactic) acid-poly(ethylene)glycol copolymer or the diblock poly(lactic)-co-poly(glycolic) acid-poly(ethylene)glycol copolymer comprises poly(ethylene) glycol having a molecular weight of about 5 to about 15 kDa, or about 4 kDa to about 6 kDa. For example, the poly(ethylene) glycol may have a number average molecule weight of about 5 kDa, 7.5 kDa, or about 10 kDa.

In an exemplary embodiment, the therapeutic nanoparticle may include about 0.1% to about 40% by weight a therapeutic agent, and 10% to about 90% by weight a diblock poly(lactic) acid-poly(ethylene)glycol copolymer, wherein the diblock poly(lactic) acid-poly(ethylene)glycol copolymer comprises poly(lactic) acid having a number average molecule weight of about 50 kDa to about 80 kDa and poly(ethylene) glycol having a number average molecule weight of about 5 kDa. In another embodiment, the therapeutic nanoparticle may include about 0.1% to about 40% by weight a therapeutic agent, and 10% to about 90% by weight a diblock poly(lactic) acid-poly(ethylene)glycol copolymer, wherein the diblock poly(lactic) acid-poly(ethylene)glycol copolymer comprises poly(lactic) acid having a number average molecule weight of about 50 kDa and poly(ethylene) glycol having a number average molecule weight of about 5 kDa. In yet another embodiment, the therapeutic nanoparticle may include about 0.1% to about 40% by weight a therapeutic agent, and 10% to about 90% by weight a diblock poly(lactic) acid-poly(ethylene)glycol copolymer, wherein the diblock poly(lactic) acid-poly(ethylene)glycol copolymer comprises poly(lactic) acid having a number average molecule weight of about 30 kDa and poly(ethylene) glycol having a number average molecule weight of about 5 kDa. In a further embodiment, the therapeutic nanoparticle may include about 1% to about 20% by weight a therapeutic agent, and 50% to about 90% by weight a diblock poly(lactic) acid-poly(ethylene)glycol copolymer or a diblock poly(lactic)-co-poly(glycolic) acid-poly(ethylene)glycol copolymer.

Also contemplated herein are therapeutic nanoparticles comprising therapeutic agents selected from vinca alkaloids, non-steroidal anti-inflammatory drugs, nitrogen mustard agents, taxanes, platinum chemotherapeutic agents, mTOR inhibitors, boronate esters or peptide boronic acid compounds, and epothilone. For example, the contemplated therapeutic nanoparticles may include therapeutic agents such as cisplatin, oxaplatin, ketorolac, rofecoxib, celecoxib, diclofenac, dihexanoate Pt(IV), vinblastine, vinorelbine, vindesine, vincristine; docetaxel, sirolimus, temsirolimus, everolimus, bortezomib, and epothilone. In an embodiment, the therapeutic agent may be docetaxel.

Compositions are provided such as compositions comprising a plurality of disclosed nanoparticles and a pharmaceutically acceptable excipient. In some embodiments, biocompatible, therapeutic polymeric nanoparticles that form part of a contemplated composition have a glass transition temperature of between about 42° C. and about 50° C., or between about 38° C. and about 42° C. In some embodiments, upon administration to a patient, biocompatible, therapeutic polymeric nanoparticles disclosed herein circulate in the plasma of the patient for at least about 24 hours e.g., about 24 hours to about 48 hours, and/or for example, upon administration to a patient, the biocompatible, therapeutic polymeric nanoparticles release the therapeutic agent in-vivo for at least 24 hours.

Also contemplated herein are methods of making disclosed nanoparticles and methods of treating cancers (for example, breast, lung, or prostate cancer) comprising administering to a patient in need thereof a disclosed particle or composition.

In another embodiment, provided herein is a controlled release therapeutic nanoparticle comprising about 0.2 to about 20 weight percent, (e.g. about 2 to about 20 weight percent) of a therapeutic agent or a pharmaceutically acceptable salt thereof, and a diblock poly(lactic) acid-poly(ethylene)glycol copolymer wherein a poly(lactic) acid block of the diblock copolymer has a number average molecule weight of about 40 kDa to about 80 kDa (e.g., about 45 to about 75 kDa, or about 40 to about 60 kDa and wherein said therapeutic agent is released at a controlled release rate. In yet another embodiment, provided herein is a controlled release therapeutic nanoparticle comprising about e.g., 0.2 to about 20 weight percent (e.g. about 2 to about 10, or about 3 to about 15 weight percent) of a therapeutic agent or a pharmaceutically acceptable salt thereof, and a diblock poly(lactic)-co-poly(glycolic) acid-poly(ethylene)glycol copolymer wherein a poly(lactic)-co-poly(glycolic) acid block of the diblock copolymer has a number average molecule weight of about 40 kDa to about 80 kDa, and wherein said therapeutic agent is released at a controlled release rate. For example, the poly(ethylene)glycol block may have a number average molecular weight of about 4 kDa to about 16 kDa, 5 kDa to about 12 kDa, or about 7.5 kDa or about 10 kDa. In one embodiment, the said controlled release therapeutic nanoparticle releases the therapeutic agent over a period of at least 1 day or more when administered to a patient. In another embodiment, the said controlled release therapeutic nanoparticle releases the therapeutic agent over a period of at least 1 day to about 4 days or more when administered to a patient.

### BRIEF DESCRIPTION OF THE DRAWINGS

FIG. 1 is a flow chart for an emulsion process for forming disclosed nanoparticles.

FIGS. 2A and 2B depict a flow diagram for a disclosed emulsion process.

FIG. 3 depicts in vitro release of docetaxel from various nanoparticles disclosed herein.

FIG. 4 depicts in vitro release of bortezomib from various nanoparticles disclosed herein.

FIG. 5 depicts in vitro release of vinorelbine from various nanoparticles disclosed herein.

FIG. 6 depicts in vitro release of vincristine o from various nanoparticles disclosed herein.

FIG. 7 depicts in vitro release of bendamustine HCl from various nanoparticles disclosed herein.

FIG. 8 depicts in vitro release of diclofenac from various nanoparticles disclosed herein.

FIG. 9 depicts in vitro release of ketorolac from various nanoparticles disclosed herein.

FIG. 10 depicts in vitro release of rofecoxib from various nanoparticles disclosed herein.

FIG. 11 depicts in vitro release of celecoxib from various nanoparticles disclosed herein, and impact of drug load.

FIG. 12 depicts in vitro release of celecoxib from various nanoparticles disclosed herein with low drug load.

### DETAILED DESCRIPTION

The present invention generally relates to polymeric nanoparticles that include a therapeutic agent or drug, and methods of making and using such therapeutic nanoparticles. In general, a “nanoparticle” refers to any particle having a diameter of less than 1000 nm, e.g. about 10 nm to about 250 nm. Disclosed therapeutic nanoparticles may include nanoparticles having a diameter of about 60 to about 190 nm, or about 70 to about 190 nm, or about 60 to about 180 nm, about 70 nm to about 180 nm, or about 50 nm to about 250 nm.

Disclosed nanoparticles may include about 0.1 to about 40 weight percent, about 0.1 to about 30 weight percent, about 0.1 to about 20 weight percent, about 0.2 to about 20 weight percent, or about 1 to about 30 weight percent of a therapeutic agent, such as an antineoplastic agent, e.g. a taxane agent (for example, docetaxel).

Nanoparticles disclosed herein include one or more biocompatible and/or biodegradable polymers, for example, a high molecular weight diblock poly(lactic) acid-poly(ethylene)glycol copolymer or a high molecular weight diblock poly(lactic)-co-poly(glycolic) acid-poly(ethylene)glycol copolymer. The diblock poly(lactic) acid-poly(ethylene)glycol copolymer comprises poly(lactic) acid having a number average molecule weight of about 30 kDa to about 90 kDa, or about 40 kDa to about 90 kDa. The diblock poly(lactic)-co-poly(glycolic) acid-poly(ethylene)glycol copolymer comprises poly(lactic)-co-poly(glycolic) acid having a number average molecule weight of about 30 kDa to about 90 kDa, or about 40 kDa to about 90 kDa. For example, a contemplated nanoparticle may include about 0.1 to about 40 weight percent of a therapeutic agent and about 10 to about 90 weight percent a diblock poly(lactic) acid-poly(ethylene)glycol copolymer, wherein the diblock poly(lactic) acid-poly(ethylene)glycol copolymer comprises poly(lactic) acid having a number average molecule weight of about 30 kDa to about 90 kDa, or about 40 kDa to about 90 kDa. In one embodiment, the poly(lactic) acid has a number average molecule weight of about 30 kDa. In another embodiment, the poly(lactic) acid has a number average molecule weight of about 50 kDa to about 80 kDa. In yet another embodiment, the poly(lactic) acid has a number average molecule weight of about 50 kDa. In some embodiments, the diblock poly(lactic) acid-poly(ethylene)glycol copolymer or the diblock poly(lactic)-co-poly(glycolic) acid-poly(ethylene)glycol copolymer comprises poly(ethylene) glycol having a molecular weight of about 4 kDa to about 6 kDa. For example, the poly(ethylene) glycol may have a number average molecule weight of about 5 kDa.

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

“Pharmaceutically or pharmacologically acceptable” describes molecular entities and compositions that do not produce an adverse, allergic or other untoward reaction when administered to an animal, or a human, as appropriate. For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

“Individual,” “patient,” or “subject” are used interchangeably and include any animal, including mammals, such as mice, rats, other rodents, rabbits, dogs, cats, swine, cattle, sheep, horses, or primates, and most preferably humans. The compounds and compositions of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). “Modulation” includes antagonism (e.g., inhibition), agonism, partial antagonism and/or partial agonism.

In the present specification, the term “therapeutically effective amount” means the amount of the subject compound or composition that will elicit the biological or medical response of a tissue, system, animal or human that is being sought by the researcher, veterinarian, medical doctor or other clinician. The compounds and compositions of the invention are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts, such as calcium, magnesium, sodium, lithium, zinc, potassium, and iron salts.

### Therapeutic Particles

Contemplated biocompatible, therapeutic polymeric nanoparticles include a biodegradable polymer, for example, a high molecular weight diblock poly(lactic) acid-poly(ethylene)glycol copolymer, wherein the diblock poly(lactic) acid-poly(ethylene)glycol copolymer comprises poly(lactic) acid having a number average molecule weight of about 30 kDa to about 90 kDa or about 40 kDa to about 90 kDa, e.g. about 45 to about 65 kDa, or about 45 to 55 kDa. Also contemplated herein are biocompatible, therapeutic polymeric nanoparticles that include a biodegradable polymer, for example, a high molecular weight diblock poly(lactic)-co-poly(glycolic) acid-poly(ethylene)glycol copolymer, wherein the diblock poly(lactic)-co-poly(glycolic) acid-poly(ethylene)glycol copolymer comprises poly(lactic)-co-poly(glycolic) acid having a number average molecule weight of about 30 kDa to about 90 kDa or about 40 kDa to about 90 kDa.

In an embodiment, a biocompatible, therapeutic polymeric nanoparticle contemplated herein includes a therapeutic agent and a high molecular weight diblock poly(lactic) acid-poly(ethylene)glycol copolymer or a diblock poly(lactic)-co-poly(glycolic) acid-poly(ethylene)glycol copolymer. In an exemplary embodiment, the particle may include about 0.1 to about 40 weight percent of a therapeutic agent (e.g. about 1 to about 20 weight percent, about 2 to about 20 weight percent, about 3 to about 6 weight percent, about 4 to about 10 weight percent, or about 6 to about 10 weight percent therapeutic agent), and about 10 to about 90 (or about 70 to about 95, or about 80 to about 99 weight percent) weight percent a diblock poly(lactic) acid-poly(ethylene)glycol copolymer, wherein the diblock poly(lactic) acid-poly(ethylene)glycol copolymer comprises poly(lactic) acid having a number average molecule weight of about 30 kDa. In another exemplary embodiment, the particle may include about 0.1 to about 40 weight percent of a therapeutic agent and about 10 to about 90 weight percent a diblock poly(lactic) acid-poly(ethylene)glycol copolymer, wherein the diblock poly(lactic) acid-poly(ethylene)glycol copolymer comprises poly(lactic) acid having a number average molecule weight of about 50 kDa. In another embodiment, the particle may include about 0.1 to about 40 weight percent of a therapeutic agent and about 10 to about 90 weight percent a diblock poly(lactic) acid-poly(ethylene)glycol copolymer, wherein the diblock poly(lactic) acid-poly(ethylene)glycol copolymer comprises poly(lactic) acid having a number average molecule weight of about 80 kDa. In certain embodiments, the diblock poly(lactic) acid-poly(ethylene)glycol copolymer or the diblock poly(lactic)-co-poly(glycolic) acid-poly(ethylene)glycol copolymer comprises poly(ethylene) glycol having a molecular weight of about 4 kDa to about 20 kDa, about 4 kDa to about 15 kDa, or about 6 kDa to about 12 kDa. For example, the poly(ethylene) glycol may have a number average molecule weight of about 5 kDa, 7.5 kDa or 10 kDa.

In some embodiments, contemplated nanoparticles may further include a biocompatible homopolymer such as poly(lactic) acid, or a polymer such as poly(lactic)-co-poly(glycolic) acid. For example, contemplated nanoparticles may further include a poly(lactic) acid or PLGA with a number average molecular weight of about 50 kDa to about 100 kDa, about 30 kDa to about 100 kDa, about 50 kDa to about 90 kDa, about 60 to about 80 kDa.

Also disclosed herein are compositions comprising a plurality of biocompatible, therapeutic polymeric nanoparticles as disclosed herein and a pharmaceutically acceptable excipient.

Disclosed nanoparticles may have a substantially spherical (i.e., the particles generally appear to be spherical), or non-spherical configuration. For instance, the particles, upon swelling or shrinkage, may adopt a non-spherical configuration.

Disclosed nanoparticles may have a characteristic dimension of less than about 1 micrometer, where the characteristic dimension of a particle is the diameter of a perfect sphere having the same volume as the particle. For example, the particle can have a characteristic dimension of the particle can be less than about 300 nm, less than about 200 nm, less than about 150 nm, less than about 100 nm, less than about 50 nm, less than about 30 nm, less than about 10 nm, less than about 3 nm, or less than about 1 nm in some cases. In particular embodiments, disclosed nanoparticles may have a diameter of about 70 nm to about 250 nm, or about 70 nm to about 180 nm, about 80 nm to about 170 nm, about 80 nm to about 130 nm.

In some embodiments, disclosed therapeutic particles and/or compositions include targeting agents such as dyes, for example Evans blue dye. Such dyes may be bound to or associated with a therapeutic particle, or disclosed compositions may include such dyes. For example, Evans blue dye may be used, which may bind or associate with albumin, e.g. plasma albumin.

Disclosed therapeutic particles, may, in some embodiments, include a targeting moiety, i.e., a moiety able to bind to or otherwise associate with a biological entity. The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. Therapeutic compositions disclosed herein may, for example, be locally administered to a designated region such as a blood vessel.

For example, disclosed herein is a therapeutic polymeric nanoparticle comprising a first non-functionalized polymer; an optional second non-functionalized polymer; an optional functionalized polymer comprising a targeting moiety; and a therapeutic agent. In an embodiment, the first non-functionalized polymer is PLA, PLGA, or PEG, or copolymers thereof, e.g. a diblock co-polymer PLA-PEG or a diblock co-polymer PLGA-PEG. For example, exemplary nanoparticle may have a PEG corona with a density of about 0.065 g/cm³, or about 0.01 to about 0.10 g/cm³.

In one set of embodiments, the particles can have an interior and a surface, where the surface has a composition different from the interior, i.e., there may be at least one compound present in the interior but not present on the surface (or vice versa), and/or at least one compound is present in the interior and on the surface at differing concentrations. For example, in one embodiment, a compound, such as a targeting moiety (i.e., a low-molecular weight ligand) of a polymeric conjugate of the present invention, may be present in both the interior and the surface of the particle, but at a higher concentration on the surface than in the interior of the particle, although in some cases, the concentration in the interior of the particle may be essentially nonzero, i.e., there is a detectable amount of the compound present in the interior of the particle.

In some cases, the interior of the particle is more hydrophobic than the surface of the particle. For instance, the interior of the particle may be relatively hydrophobic with respect to the surface of the particle, and a drug or other payload may be hydrophobic, and readily associates with the relatively hydrophobic center of the particle. The drug or other payload can thus be contained within the interior of the particle, which can shelter it from the external environment surrounding the particle (or vice versa). For instance, a drug or other payload contained within a particle administered to a subject will be protected from a subject's body, and the body may also be substantially isolated from the drug for at least a period of time.

Disclosed nanoparticles may be stable, for example in a solution that may contain a saccharide, for at least about 24 hours, about 2 days, 3 days, about 4 days or at least about 5 days at room temperature, or at 25° C.

Nanoparticles disclosed herein may have controlled release properties, e.g., may be capable of delivering an amount of active agent to a patient, e.g., to specific site in a patient, over an extended period of time, e.g. over 1 day, 1 week, or more. For example, provided herein is a controlled release therapeutic nanoparticle comprising about 0.2 to about 20 weight percent of a therapeutic agent or a pharmaceutically acceptable salt thereof, and a diblock poly(lactic) acid-poly(ethylene)glycol copolymer, wherein a poly(lactic) acid block of the diblock copolymer has a number average molecule weight of about 40 kDa to about 60 kDa, and wherein said therapeutic agent is released at a controlled release rate. Also provided herein is a controlled release therapeutic nanoparticle comprising about 0.2 to about 20 weight percent of a therapeutic agent or a pharmaceutically acceptable salt thereof, and a diblock poly(lactic)-co-poly(glycolic) acid-poly(ethylene)glycol copolymer, wherein a poly(lactic)-co-poly(glycolic) acid block of the diblock copolymer has a number average molecule weight of about 40 kDa to about 60 kDa, and wherein said therapeutic agent is released at a controlled release rate. In one embodiment, the said controlled release therapeutic nanoparticle releases the therapeutic agent over a period of at least 1 day or more when administered to a patient. In another embodiment, the said controlled release therapeutic nanoparticle releases the therapeutic agent over a period of at least 1 day to about 4 days or more when administered to a patient.

For example, upon administering to a patient (e.g. systemically (e.g. intravenously or subcutaneously), disclosed nanoparticles may circulate in the plasma of the patient for at least 24 hours (e.g. about 18 hours to about 48 hours, or about 24 hours to about 36 hours), and may release the therapeutic agent over a period of 24 hours or more, e.g. over a period of about 1 day, 2 days, 24-36 hours.

In one embodiment, the invention comprises a nanoparticle comprising 1) a polymeric matrix and 2) an amphiphilic compound or layer that surrounds or is dispersed within the polymeric matrix forming a continuous or discontinuous shell for the particle. An amphiphilic layer can reduce water penetration into the nanoparticle, thereby enhancing drug encapsulation efficiency and slowing drug release. Further, these amphiphilic layer protected nanoparticles can provide therapeutic advantages by releasing the encapsulated drug and polymer at appropriate times.

As used herein, the term “amphiphilic” refers to a property where a molecule has both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. Exemplary amphiphilic compound include, for example, one or a plurality of the following: naturally derived lipids, surfactants, or synthesized compounds with both hydrophilic and hydrophobic moieties.

Specific examples of amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

In a particular embodiment, an amphiphilic component may include lecithin, and/or in particular, phosphatidylcholine.

### Polymers

Contemplated herein are nanoparticles comprising high molecular weight polymers, for example, high molecular weight copolymers. In one embodiment, the molecular weight of the polymer can be optimized for effective treatment as disclosed herein. For example, the weight of a polymer may influence particle degradation rate, solubility, water uptake, and drug release kinetics. The molecular weight of the polymer can be adjusted such that the particle biodegrades in the subject being treated within a reasonable period of time (ranging from a few hours to 1-2 weeks, 3-4 weeks, 5-6 weeks, 7-8 weeks, etc.) For example, a disclosed particle may comprise a copolymer of PLA and PEG or PLGA and PEG, wherein the PLA or PLGA portion may have a number average molecule weight of about 30 kDa to about 90 kDa or about 40 kDa to about 90 kDa, and the PEG portion may have a molecular weight of about 4 kDa to about 6 kDa. In an exemplary embodiment, the PLA or the PLGA portion may have a number average molecule weight of 30 kDa, 50 kDa, 55 kDa, 47 kDa, 65 kDa, or 80 kDa. The PEG portion may have a molecular weight of about 2.5 kDa to about 20 Da, e.g. about 5 to about 15 kDa, or about 5 kDa, 7.5 kDa, 10 kDa.

A wide variety of high molecular weight polymers and methods for forming particles therefrom are known in the art of drug delivery. Disclosed nanoparticles may include one or more high molecular weight polymers, e.g. a first polymer that may be a co-polymer, e.g. a diblock co-polymer, and optionally a polymer that may be for example a homopolymer. In some embodiments, disclosed nanoparticles include a matrix of polymers. Disclosed therapeutic nanoparticles may include a therapeutic agent that can be associated with the surface of, encapsulated within, surrounded by, and/or dispersed throughout a polymeric matrix.

Any high molecular weight polymer can be used in accordance with the present invention. Such polymers can be natural or unnatural (synthetic) polymers. Polymers can be homopolymers or copolymers comprising two or more monomers. In terms of sequence, copolymers can be random, block, or comprise a combination of random and block sequences. Contemplated polymers may be biocompatible and/or biodegradable.

Disclosed particles can include high molecular weight copolymers, which, in some embodiments, describes two or more polymers (such as those described herein) that have been associated with each other, usually by covalent bonding of the two or more polymers together.

Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In some embodiments, the high molecular weight polymer (e.g., copolymer, e.g., block copolymer) can be amphiphilic, i.e., having a hydrophilic portion and a hydrophobic portion, or a relatively hydrophilic portion and a relatively hydrophobic portion. A hydrophilic polymer can be one generally that attracts water and a hydrophobic polymer can be one that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about 60°). In some cases, the hydrophilicity of two or more polymers may be measured relative to each other, i.e., a first polymer may be more hydrophilic than a second polymer. For instance, the first polymer may have a smaller contact angle than the second polymer.

In one set of embodiments, a high molecular weight polymer (e.g., copolymer, e.g., block copolymer) contemplated herein includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the therapeutic particles contemplated herein can be non-immunogenic. The term non-immunogenic as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments of the present invention include polydioxanone (PDO), polyhydroxyalkanoate, polyhydroxybutyrate, poly(glycerol sebacate), polyglycolide, polylactide, PLGA, PLA, polycaprolactone, or copolymers or derivatives including these and/or other polymers.

In certain embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

For instance, a contemplated polymer may be one that hydrolyzes spontaneously upon exposure to water (e.g., within a subject), the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

In some embodiments, polymers may be polyesters, including copolymers comprising lactic acid and glycolic acid units, such as poly(lactic)-co-poly(glycolic) acid, poly(lactic acid-co-glycolic acid), and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids or polyanhydrides.

In other embodiments, contemplated polyesters for use in disclosed nanoparticles may be diblock copolymers, e.g., PEGylated polymers and copolymers (containing poly(ethylene glycol) repeat units) such as of lactide and glycolide (e.g., PEGylated PLA, PEGylated PGA, PEGylated PLGA), PEGylated poly(caprolactone), and derivatives thereof. For example, a “PEGylated” polymer may assist in the control of inflammation and/or immunogenicity (i.e., the ability to provoke an immune response) and/or lower the rate of clearance from the circulatory system via the reticuloendothelial system (RES), due to the presence of the poly(ethylene glycol) groups.

PEGylation may also be used, in some cases, to decrease charge interaction between a polymer and a biological moiety, e.g., by creating a hydrophilic layer on the surface of the polymer, which may shield the polymer from interacting with the biological moiety. In some cases, the addition of poly(ethylene glycol) repeat units may increase plasma half-life of the polymer (e.g., copolymer, e.g., block copolymer), for instance, by decreasing the uptake of the polymer by the phagocytic system while decreasing transfection/uptake efficiency by cells. Those of ordinary skill in the art will know of methods and techniques for PEGylating a polymer, for example, by using EDC (1-ethyl-3-(3-dimethylaminopropyl) carbodiimide hydrochloride) and NHS (N-hydroxysuccinimide) to react a polymer to a PEG group terminating in an amine, by ring opening polymerization techniques (ROMP), or the like.

Other contemplated polymers that may form part of a disclosed nanoparticle may include poly(ortho ester) PEGylated poly(ortho ester), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], and derivatives thereof. In other embodiments, polymers can be degradable polyesters bearing cationic side chains. Examples of these polyesters include poly(L-lactide-co-L-lysine), poly(serine ester), poly(4-hydroxy-L-proline ester).

In other embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

PLGA contemplated for use as described herein can be characterized by a lactic acid:glycolic acid ratio of e.g., approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., a PLGA block copolymer or PLGA-PEG block copolymer), may be selected to optimize for various parameters such as water uptake, therapeutic agent release and/or polymer degradation kinetics can be optimized. In other embodiments, the end group of a PLA polymer chain may be a carboxylic acid group, an amine group, or a capped end group with e.g., a long chain alkyl group or cholesterol.

Particles disclosed herein may or may not contain PEG. In addition, certain embodiments can be directed towards copolymers containing poly(ester-ether)s, e.g., polymers having repeat units joined by ester bonds (e.g., R—C(O)—O—R′ bonds) and/or ether bonds (e.g., R—O—R′ bonds). Contemplated herein, in certain embodiments, is a biodegradable polymer, such as a hydrolyzable polymer containing carboxylic acid groups, that may be conjugated with poly(ethylene glycol) repeat units to form a poly(ester-ether).

In one embodiment, a disclosed nanoparticle has a glass transition temperature, e.g. in a disclosed aqueous solution, may be about 37° C. to about 39° C., or about 37° C. to about 38° C. In another embodiment, an aqueous suspension of nanoparticles may have a glass transition temperature that may be about 38° C. to about 42° C. (e.g., about 39° C. to about 41° C.), or may be about 42° C. to about 50° C. (e.g. about 41-45° C., e.g. for slow release particles). The glass transition temperature may be measured by Heat Flux Differential Scanning calorimetry or Power Compensation Differential Scanning calorimetry.

In certain embodiments, one or more polymers of a disclosed particle may be conjugated to a lipid. The polymer may be, for example, a lipid-terminated PEG. As described below, the lipid portion of the polymer can be used for self assembly with another polymer, facilitating the formation of a particle. For example, a hydrophilic polymer could be conjugated to a lipid that will self assemble with a hydrophobic polymer.

In some embodiments, lipids can be oils. In general, any oil known in the art can be conjugated to the polymers used in the invention. In some embodiments, an oil may comprise one or more fatty acid groups or salts thereof. In some embodiments, a fatty acid group may comprise digestible, long chain (e.g., C₈-C₅₀), substituted or unsubstituted hydrocarbons. In some embodiments, a fatty acid group may be a C₁₀-C₂₀ fatty acid or salt thereof. In some embodiments, a fatty acid group may be a C₁₅-C₂₀ fatty acid or salt thereof. In some embodiments, a fatty acid may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid.

In one embodiment, the lipid can be of the Formula V:

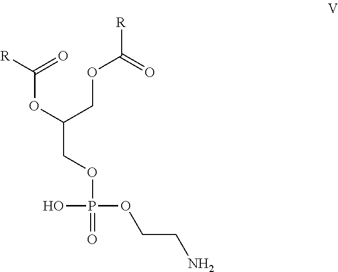

and salts thereof, wherein each R is, independently, C₁₋₃₀ alkyl. In one embodiment of Formula V, the lipid can be 1,2 distearoyl-sn-glycero-3-phosphoethanolamine (DSPE), and salts thereof, e.g., the sodium salt.

### Preparation of Nanoparticles

Another aspect of the invention is directed to systems and methods of making disclosed nanoparticles.

In an embodiment, provided herein is a method of preparing a plurality of biocompatible, therapeutic polymeric nanoparticles comprising: combining a therapeutic agent (e.g. docetaxel or bortezomib), and a biodegradable high molecular weight copolymer (e.g. PLA-PEG or PLGA-PEG), with an organic solution to form a first organic phase; combining the first organic phase with a first aqueous solution to form a second phase; emulsifying the second phase to form an emulsion phase; adding a drug solubilizer to the emulsion phase to form a solubilized phase; and recovering the biocompatible, therapeutic polymeric nanoparticles.

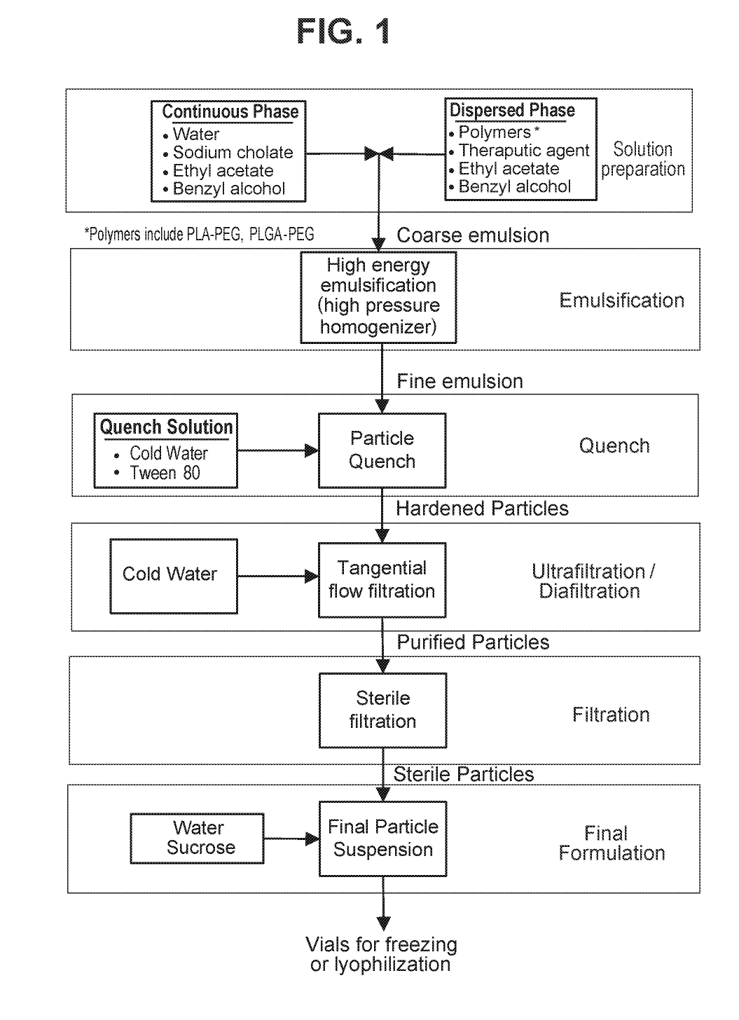

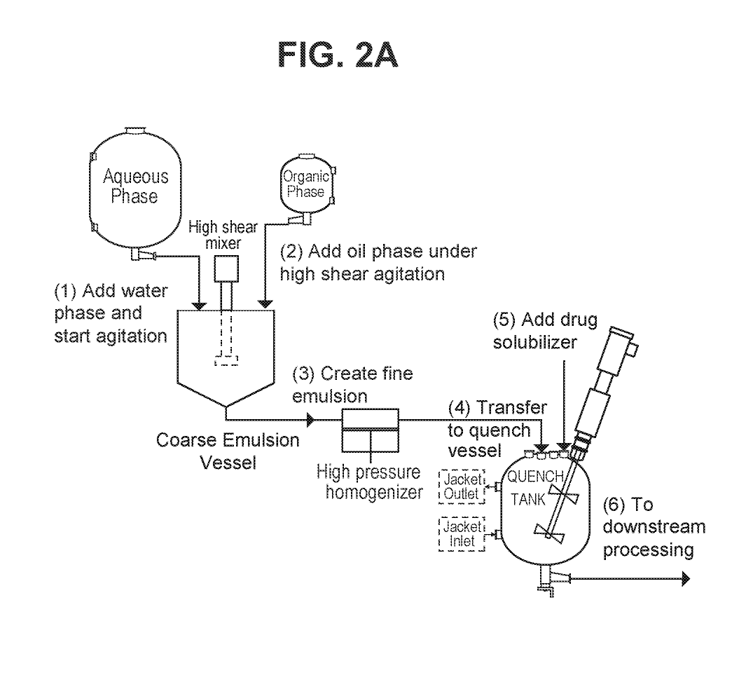

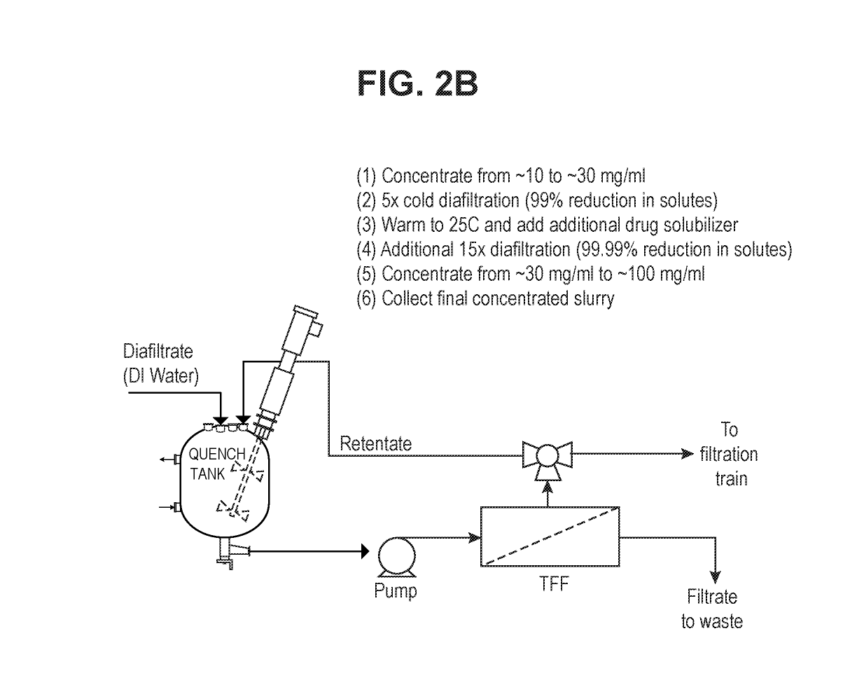

In an embodiment, a nanoemulsion process is provided, such as the process represented in FIGS. 1 and 2. For example, a therapeutic agent, and a high molecular weight co-polymer (for example, PLA-PEG or PLGA-PEG), is mixed with an organic solution to form a first organic phase. Such first phase may include about 5 to about 50% weight solids, e.g. about 5 to about 40% solids, or about 10 to about 30% solids, e.g. about 10%, 15%, 20% solids. The first organic phase may be combined with a first aqueous solution to form a second phase. The organic solution can include, for example, acetonitrile, tetrahydrofuran, ethyl acetate, isopropyl alcohol, isopropyl acetate, dimethylformamide, methylene chloride, dichloromethane, chloroform, acetone, benzyl alcohol, Tween 80, Span 80, or the like, and combinations thereof. In an embodiment, the organic phase may include benzyl alcohol, ethyl acetate, and combinations thereof. The second phase can be between about 1 and 50 weight %, e.g., 5-40 weight %, solids. The aqueous solution can be water, optionally in combination with one or more of sodium cholate, ethyl acetate, and benzyl alcohol.

For example, the oil or organic phase may use solvent that is only partially miscible with the nonsolvent (water). Therefore, when mixed at a low enough ratio and/or when using water pre-saturated with the organic solvents, the oil phase remains liquid. The oil phase may be emulsified into an aqueous solution and, as liquid droplets, sheared into nanoparticles using, for example, high energy dispersion systems, such as homogenizers or sonicators. The aqueous portion of the emulsion, otherwise known as the “water phase”, may be a surfactant solution consisting of sodium cholate and pre-saturated with ethyl acetate and benzyl alcohol.

Emulsifying the second phase to form an emulsion phase may be performed in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g. probe sonicator or a high pressure homogenizer, e.g. by using 1, 2, 3 or more passes through a homogenizer. For example, when a high pressure homogenizer is used, the pressure used may be about 4000 to about 8000 psi, or about 4000 to about 5000 psi, e.g. 4000 or 5000 psi.

Either solvent evaporation or dilution may be needed to complete the extraction of the solvent and solidify the particles. For better control over the kinetics of extraction and a more scalable process, a solvent dilution via aqueous quench may be used. For example, the emulsion can be diluted into cold water to a concentration sufficient to dissolve all of the organic solvent to form a quenched phase. Quenching may be performed at least partially at a temperature of about 5° C. or less. For example, water used in the quenching may be at a temperature that is less that room temperature (e.g. about 0 to about 10° C., or about 0 to about 5° C.).

In some embodiments, not all of the therapeutic agent is encapsulated in the particles at this stage, and a drug solubilizer is added to the quenched phase to form a solubilized phase. The drug solubilizer may be for example, Tween 80, Tween 20, polyvinyl pyrrolidone, cyclodextran, sodium dodecyl sulfate, or sodium cholate. For example, Tween-80 may added to the quenched nanoparticle suspension to solubilize the free drug and prevent the formation of drug crystals. In some embodiments, a ratio of drug solubilizer to therapeutic agent is about 100:1 to about 10:1.

The solubilized phase may be filtered to recover the nanoparticles. For example, ultrafiltration membranes may be used to concentrate the nanoparticle suspension and substantially eliminate organic solvent, free drug, and other processing aids (surfactants). Exemplary filtration may be performed using a tangential flow filtration system. For example, by using a membrane with a pore size suitable to retain nanoparticles while allowing solutes, micelles, and organic solvent to pass, nanoparticles can be selectively separated. Exemplary membranes with molecular weight cut-offs of about 300-500 kDa (˜5-25 nm) may be used.

Diafiltration may be performed using a constant volume approach, meaning the diafiltrate (cold deionized water, e.g. about 0° C. to about 5° C., or 0 to about 10° C.) may be added to the feed suspension at the same rate as the filtrate is removed from the suspension. In some embodiments, filtering may include a first filtering using a first temperature of about 0° C. to about 5° C., or 0° C. to about 10° C., and a second temperature of about 20° C. to about 30° C., or 15° C. to about 35° C. For example, filtering may include processing about 1 to about 6 diavolumes at about 0° C. to about 5° C., and processing at least one diavolume (e.g. about 1 to about 3 or about 1-2 diavolumes) at about 20° C. to about 30° C.

After purifying and concentrating the nanoparticle suspension, the particles may be passed through one, two or more sterilizing and/or depth filters, for example, using ˜0.2 μm depth pre-filter.

In exemplary embodiment of preparing nanoparticles, an organic phase is formed composed of a mixture of a therapeutic agent, e.g., docetaxel or bortezomib, and a high molecular copolymer (e.g. PLA-PEG or PLGA-PEG). The organic phase may be mixed with an aqueous phase at approximately a 1:5 ratio (oil phase:aqueous phase) where the aqueous phase is composed of a surfactant and optionally dissolved solvent. A primary emulsion may then formed by the combination of the two phases under simple mixing or through the use of a rotor stator homogenizer. The primary emulsion is then formed into a fine emulsion through the use of e.g. high pressure homogenizer. Such fine emulsion may then quenched by, e.g. addition to deionized water under mixing. An exemplary quench:emulsion ratio may be about approximately 8:1. A solution of Tween (e.g., Tween 80) can then be added to the quench to achieve e.g. approximately 2% Tween overall, which may serve to dissolve free, unencapsulated drug. Formed nanoparticles may then be isolated through either centrifugation or ultrafiltration/diafiltration.

### Therapeutic Agents

According to the present invention, any agents including, for example, therapeutic agents (e.g. anti-cancer agents), diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.) may be delivered by the disclosed nanoparticles. Exemplary agents to be delivered in accordance with the present invention include, but are not limited to, small molecules (e.g. cytotoxic agents), nucleic acids (e.g., siRNA, RNAi, and mircoRNA agents), proteins (e.g. antibodies), peptides, lipids, carbohydrates, hormones, metals, radioactive elements and compounds, drugs, vaccines, immunological agents, etc., and/or combinations thereof. In some embodiments, the agent to be delivered is an agent useful in the treatment of cancer (e.g., breast, lung, or prostate cancer).

Disclosed therapeutic nanoparticles may comprise about 0.1 to about 40 weight percent of a therapeutic agent, e.g. about 1 to about 15 weight percent, e.g. about 3 to about 10 weight percent (e.g. about 3 to about 6 weigh percent) e.g. about 2 to about 20 (e.g. about 6 to about 10 weight percent) or about 3 to about 15, or about 4 to about 12 weight percent therapeutic agent.

The active agent or drug may be a therapeutic agent such as mTorr inhibitors (e.g., sirolimus, temsirolimus, or everolimus), vinca alkaloids (e.g. vinorelbine or vincristine), a diterpene derivative, a taxane (e.g. paclitaxel or its derivatives such as DHA-paclitaxel or PG-paxlitaxelor, or docetaxel), a boronate ester or peptide boronic acid compound (e.g. bortezomib), a cardiovascular agent (e.g. a diuretic, a vasodilator, angiotensin converting enzyme, a beta blocker, an aldosterone antagonist, or a blood thinner), a corticosteroid, an antimetabolite or antifolate agent (e.g. methotrexate), a chemotherapeutic agent (e.g. epothilone B), a nitrogen mustard agent (e.g. bendamustine), or the active agent or drug may be an siRNA.

In one set of embodiments, the payload is a drug or a combination of more than one drug. Such particles may be useful, for example, in embodiments where a targeting moiety may be used to direct a particle containing a drug to a particular localized location within a subject, e.g., to allow localized delivery of the drug to occur. Exemplary therapeutic agents include chemotherapeutic agents such as doxorubicin (adriamycin), gemcitabine (gemzar), daunorubicin, procarbazine, mitomycin, cytarabine, etoposide, methotrexate, 5-fluorouracil (5-FU), vinca alkaloids such as vinblastine, vinoelbine, vindesine, or vincristine; bleomycin, taxanes such as paclitaxel (taxol) or docetaxel (taxotere), mTOR inhibitors such as sirolimus, temsirolimus, or everolimus, aldesleukin, asparaginase, boronate esters or peptide boronic acid compounds such as bortezomib, busulfan, carboplatin, cladribine, camptothecin, CPT-11, 10-hydroxy-7-ethylcamptothecin (SN38), dacarbazine, S-I capecitabine, ftorafur, 5′deoxyflurouridine, UFT, eniluracil, deoxycytidine, 5-azacytosine, 5-azadeoxycytosine, allopurinol, 2-chloroadenosine, trimetrexate, aminopterin, methylene-10-deazaaminopterin (MDAM), oxaplatin, picoplatin, tetraplatin, satraplatin, platinum-DACH, dihexanoate platinum (IV), ormaplatin, CI-973, JM-216, epirubicin, etoposide phosphate, 9-aminocamptothecin, 10,11-methylenedioxycamptothecin, karenitecin, 9-nitrocamptothecin, TAS 103, L-phenylalanine mustard, ifosphamidemefosphamide, perfosfamide, trophosphamide carmustine, semustine, bendamustine, epothilones A-E, tomudex, 6-mercaptopurine, 6-thioguanine, amsacrine, karenitecin, acyclovir, valacyclovir, ganciclovir, amantadine, rimantadine, lamivudine, zidovudine, bevacizumab, trastuzumab, rituximab, budesonide, and combinations thereof, or the therapeutic agent may be an siRNA.

Non-limiting examples of potentially suitable drugs include anti-cancer agents, including, for example, docetaxel, mitoxantrone, and mitoxantrone hydrochloride. In another embodiment, the payload may be an anti-cancer drug such as 20-epi-1, 25 dihydroxyvitamin D3, 4-ipomeanol, 5-ethynyluracil, 9-dihydrotaxol, abiraterone, acivicin, aclarubicin, acodazole hydrochloride, acronine, acylfiilvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizdng morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisazuidinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caraceraide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castanosperrnine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethyhiorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflomithine, eflomithine hydrochloride, elemene, elsarnitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ihnofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatm, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide, lobaplatin, lombricine, lometrexol, lometrexol sodium, lomustine, lonidamine, losoxantrone, losoxantrone hydrochloride, lovastatin, loxoribine, lurtotecan, lutetium texaphyrin lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C uihibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazorurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinarnide, RNAi, rogletimide, rohitukine, romurtide, roquinimex, rubiginone Bl, ruboxyl, safingol, safingol hydrochloride, saintopin, sarcnu, sarcophytol A, sargramostim, SDI1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosafe sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine or vinorelbine tartrate, vinrosidine sulfate, vinxaltine, vinzolidine sulfate, vitaxin, vorozole, zanoterone, zeniplatin, zilascorb, zinostatin, zinostatin stimalam

The active agent or drug may be an NSAID or a pharmaceutically acceptable salt thereof. For example, the NSAID may be an acetic acid derivative, a propionic acid derivative, a salicylate, a selective COX-2 inhibitor, a sulphonanilides, a fenamic acid derivative, or an enolic acid derivative. Non-limiting examples of NSAIDs include diclofenac, ketorolac, aspirin, diflunisal, salsalate, ibuprofen, naproxen, fenoprofen, ketoprofen, flurbiprofen, oxaprozin, loxoprofen, indomethacin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, and licofelone.

In an embodiment, an active agent may (or in another embodiment, may not be) conjugated to e.g. a disclosed hydrophobic polymer that forms part of a disclosed nanoparticle, e.g an active agent such as an NSAID may be conjugated (e.g. covalently bound, e.g. directly or through a linking moiety such as linking moiety comprising e.g., —NH-alkylene-C(O)—, —NH— alkylene-O-alkylene-C(O)—, —NH-alkylene-C(O)—O-alkylene-C(O)—, or —NH-alkylene-S—) to PLA or PGLA, or a PLA or PLGA portion of a copolymer such as PLA-PEG or PLGA-PEGer, or zorubicin hydrochloride.

### Compositions and Methods of Treatment

Nanoparticles disclosed herein may be combined with pharmaceutical acceptable carriers to form a pharmaceutical composition. As would be appreciated by one of skill in this art, the carriers may be chosen based on the route of administration as described below, the location of the target issue, the drug being delivered, the time course of delivery of the drug, etc.

The pharmaceutical compositions and particles disclosed herein can be administered to a patient by any means known in the art including oral and parenteral routes. The term “patient,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, disclosed nanoparticles may be administered to a subject in need thereof systemically, e.g., by IV infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Disclosed nanoparticles may be formulated in dosage unit form for ease of administration and uniformity of dosage. The expression “dosage unit form” as used herein refers to a physically discrete unit of nanoparticle appropriate for the patient to be treated. For any nanoparticle, the therapeutically effective dose can be estimated initially either in cell culture assays or in animal models, usually mice, rabbits, dogs, or pigs. An animal model may also be used to achieve a desirable concentration range and route of administration. Such information can then be used to determine useful doses and routes for administration in humans. Therapeutic efficacy and toxicity of nanoparticles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies can be used in formulating a range of dosage for human use.

In an exemplary embodiment, a pharmaceutical composition is disclosed that includes a plurality of nanoparticles each comprising a therapeutic agent and a pharmaceutically acceptable excipient.

In some embodiments, a composition suitable for freezing is contemplated, including nanoparticles disclosed herein and a solution suitable for freezing, e.g., a sugar (e.g. sucrose) solution is added to a nanoparticle suspension. The sucrose may, e.g., act as a cryoprotectant to prevent the particles from aggregating upon freezing. For example, provided herein is a nanoparticle formulation comprising a plurality of disclosed nanoparticles, sucrose and water; wherein, for example, the nanoparticles/sucrose/water are present at about 5-10%/10-15%/80-90% (w/w/w).

In some embodiments, therapeutic particles disclosed herein may be used to treat, alleviate, ameliorate, relieve, delay onset of, inhibit progression of, reduce severity of, and/or reduce incidence of one or more symptoms or features of a disease, disorder, and/or condition. For example, disclosed therapeutic particles, that include taxane, e.g., docetaxel, may be used to treat cancers such as breast, lung, or prostate cancer in a patient in need thereof. Other types of tumors and cancer cells to be treated with therapeutic particles of the present invention include all types of solid tumors, such as those which are associated with the following types of cancers: lung, squamous cell carcinoma of the head and neck (SCCHN), pancreatic, colon, rectal, esophageal, prostate, breast, ovarian carcinoma, renal carcinoma, lymphoma and melanoma. The tumor can be associated with cancers of (i.e., located in) the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid) and is not necessarily the primary tumor. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, non-small cell lung carcinoma. Tumors can arise in the uterine cervix, uterine corpus, ovary vulva, vagina, prostate, testis, and penis, which make up the male and female genital systems, and the urinary bladder, kidney, renal pelvis, and ureter, which comprise the urinary system.

Disclosed methods for the treatment of cancer (e.g. breast or prostate cancer) may comprise administering a therapeutically effective amount of the disclosed therapeutic particles to a subject in need thereof, in such amounts and for such time as is necessary to achieve the desired result. In certain embodiments of the present invention a “therapeutically effective amount” is that amount effective for treating, alleviating, ameliorating, relieving, delaying onset of, inhibiting progression of, reducing severity of, and/or reducing incidence of one or more symptoms or features of e.g. a cancer being treated.

Also provided herein are therapeutic protocols that include administering a therapeutically effective amount of an disclosed therapeutic particle to a healthy individual (i.e., a subject who does not display any symptoms of cancer and/or who has not been diagnosed with cancer). For example, healthy individuals may be “immunized” with an inventive targeted particle prior to development of cancer and/or onset of symptoms of cancer; at risk individuals (e.g., patients who have a family history of cancer; patients carrying one or more genetic mutations associated with development of cancer; patients having a genetic polymorphism associated with development of cancer; patients infected by a virus associated with development of cancer; patients with habits and/or lifestyles associated with development of cancer; etc.) can be treated substantially contemporaneously with (e.g., within 48 hours, within 24 hours, or within 12 hours of) the onset of symptoms of cancer. Of course individuals known to have cancer may receive inventive treatment at any time.

In other embodiments, disclosed nanoparticles may be used to inhibit the growth of cancer cells, e.g., breast cancer cells. As used herein, the term “inhibits growth of cancer cells” or “inhibiting growth of cancer cells” refers to any slowing of the rate of cancer cell proliferation and/or migration, arrest of cancer cell proliferation and/or migration, or killing of cancer cells, such that the rate of cancer cell growth is reduced in comparison with the observed or predicted rate of growth of an untreated control cancer cell. The term “inhibits growth” can also refer to a reduction in size or disappearance of a cancer cell or tumor, as well as to a reduction in its metastatic potential. Preferably, such an inhibition at the cellular level may reduce the size, deter the growth, reduce the aggressiveness, or prevent or inhibit metastasis of a cancer in a patient. Those skilled in the art can readily determine, by any of a variety of suitable indicia, whether cancer cell growth is inhibited.

Inhibition of cancer cell growth may be evidenced, for example, by arrest of cancer cells in a particular phase of the cell cycle, e.g., arrest at the G2/M phase of the cell cycle. Inhibition of cancer cell growth can also be evidenced by direct or indirect measurement of cancer cell or tumor size. In human cancer patients, such measurements generally are made using well known imaging methods such as magnetic resonance imaging, computerized axial tomography and X-rays. Cancer cell growth can also be determined indirectly, such as by determining the levels of circulating carcinoembryonic antigen, prostate specific antigen or other cancer-specific antigens that are correlated with cancer cell growth. Inhibition of cancer growth is also generally correlated with prolonged survival and/or increased health and well-being of the subject.

### EXAMPLES

The invention now being generally described, it will be more readily understood by reference to the following examples which are included merely for purposes of illustration of certain aspects and embodiments of the present invention, and are not intended to limit the invention in any way.

### Example 1 Preparation of PLA-PEG

The synthesis is accomplished by ring opening polymerization of d,l-lactide with α-hydroxy-ω-methoxypoly(ethylene glycol) as the macro-initiator, and performed at an elevated temperature using Tin (II) 2-Ethyl hexanoate as a catalyst, as shown below (PEG Mₙ≈5,000 Da; PLA Mₙ≈16,000 Da; PEG-PLA Mₙ≈21,000 Da).

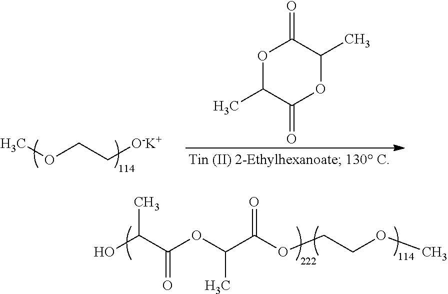

The polymer is purified by dissolving the polymer in dichloromethane, and precipitating it in a mixture of hexane and diethyl ether. The polymer recovered from this step is dried in an oven.

### Example 2 Docetaxel Nanoparticles

Docetaxel nanoparticles comprising various PLA-PEG copolymers are prepared using the following formulation: 10% (w/w) theoretical drug and 90% (w/w) polymer-PEG (16-5, 30-5, 50-5, or 80-5 PLA-PEG). % otal solids=30%. Solvents used are 21% benzyl alcohol and 79% ethyl acetate (w/w). For a 1 gram batch size, 100 mg of drug is mixed with 900 mg of polymer-PEG (16-5, 30-5, 50-5, or 80-5 PLA-PEG).

Docetaxel nanoparticles are produced as follows. In order to prepare a drug/polymer solution, appropriate amounts of docetaxel, and polymer are added to a glass vial along with appropriate amounts of ethyl acetate and benzyl alcohol. The mixture is vortexed until the drug and polymer are completely dissolved.

An aqueous solution is prepared. The 16-5 PLA-PEG formulation contains 0.5% sodium cholate, 2% benzyl alcohol, and 4% ethyl acetate in water. The 30-5 PLA-PEG formulation contains 5% sodium cholate, 2% benzyl alcohol, and 4% ethyl acetate in water, % total solids=20%. The 50-5 PLA-PEG formulation contains 5% sodium cholate, 2% benzyl alcohol, and 4% ethyl acetate in water, % total solids=20%. The 80-5 PLA-PEG formulation contains 5% sodium cholate, 2% benzyl alcohol, and 4% ethyl acetate in water, % total solids=20%. When higher molecular weight polymer-PEG is used (i.e. 30-5, 50-5, or 80-5 PLA-PEG), the concentration of sodium cholate surfactant in the water phase is increased from 0.5% to 5% in order to obtain nanoparticles with sizes similar to those particles comprising 16-5 PLA-PEG. Specifically, appropriate amounts of sodium cholate and DI water are added to a bottle and mixed using a stir plate until they are dissolved. Subsequently, appropriate amounts of benzyl alcohol and ethyl acetate are added to the sodium cholate/water mixture and mixed using a stir plate until all are dissolved.

An emulsion is formed by combining the organic phase into the aqueous solution at a ratio of 5:1 (aqueous phase:oil phase). The organic phase is poured into the aqueous solution and homogenized using hand homogenizer at room temperature to form a coarse emulsion. The solution is subsequently fed through a high pressure homogenizer (110S) to form a nanoemulsion.

The emulsion is quenched into cold DI water at <5° C. while stirring on a stir plate. The ratio of Quench to Emulsion is 8:1. Tween 80 in water is then added to the quenched emulsion at a ratio of 25:1 (Tween 80:drug).

The nanoparticles are concentrated through tangential flow filtration (TFF) followed by diafiltration to remove solvents, unencapsulated drug and solubilizer. A quenched emulsion is initially concentrated through TFF using a 300 KDa Pall cassette (2 membrane) to an approximately 100 mL volume. This is followed by diafiltration using approximately 20 diavolumes (2 L) of cold DI water. The volume is minimized by adding 100 mL of cold water to the vessel and pumping through the membrane for rinsing. Approximately 100-180 mL of material are collected in a glass vial. The nanoparticles are further concentrated using a smaller TFF to a final volume of approximately 10-20 mL.

In order to determine the solids concentration of unfiltered final slurry, a volume of final slurry is added to a tared 20 mL scintillation vial and dried under vacuum on lyo/oven. Subsequently the weight of nanoparticles is determined in the volume of the dried down slurry. Concentrated sucrose (0.666 g/g) is added to the final slurry sample to attain a final concentration of 10% sucrose.

In order to determine the solids concentration of a 0.45 μm filtered final slurry, a portion of the final slurry sample is filtered before the addition of sucrose using a 0.45 μm syringe filter. A volume of the filtered sample is then added to a tared 20 mL scintillation vial and dried under vacuum on lyo/oven. The remaining sample of unfiltered final slurry is frozen with sucrose.

Table A provides the particle size and drug load of the docetaxel nanoparticles produced as described above.

TABLE A

| Polymer | Load DTXL % | Size (nm) |
|---|---|---|
| 16/5 PLA/PEG | 4.05 | 110.70 |
| 30/5 PLA/PEG | 1.48 | 129.00 |
| 50/5 PLA/PEG | 2.75 | 170.60 |
| 80/5 PLA/PEG | 3.83 | 232.00 |

As shown in Table A, docetaxel nanoparticles comprising 50-5 PLA-PEG and 80-5 PLA-PEG result in a drug load of about 2.75% and 3.83%, respectively.

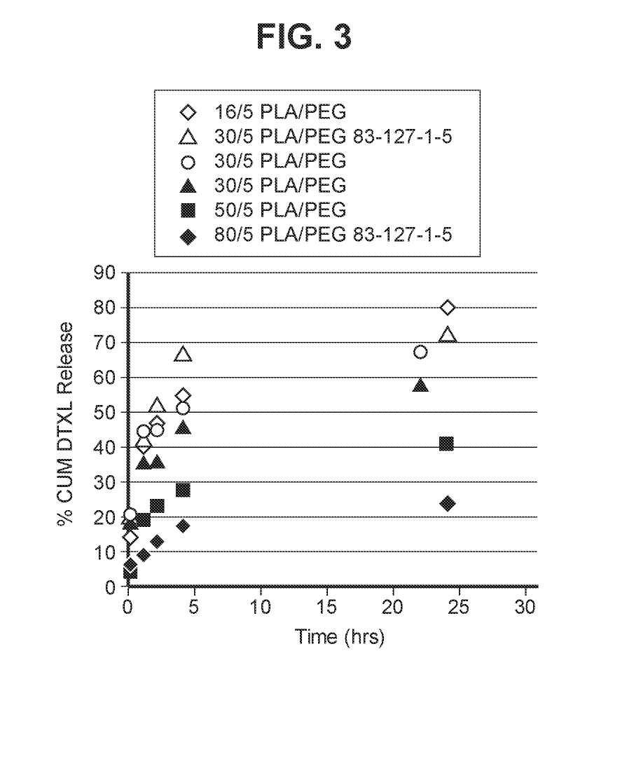

In vitro release test is performed on the above described docetaxel nanoparticles. As depicted in FIG. 3, incorporation of 50-5 PLA-PEG or 80-5 PLA-PEG slowed down the release of docetaxel from the nanoparticles compared with nanoparticles having lower molecular PLA-PEG.

### Example 3 Bortezomib Nanoparticles

Bortezomib nanoparticles comprising various PLA-PEG copolymers are prepared using the following formulation: 30% (w/w) theoretical drug and 70% (w/w) polymer-PEG (16/5, 30-5, 50-5, 65-5, or 80-5 PLA-PEG). % Total solids=20%. Solvents used are 21% benzyl alcohol and 79% ethyl acetate (w/w). For a 1 gram batch size, 300 mg of drug is mixed with 700 mg of polymer-PEG (16/5, 30-5, 50-5, 65-5, or 80-5 PLA-PEG).

Bortezomib nanoparticles are prepared using a protocol similar to the protocol described above for docetaxel nanoparticles.

Table B provides the particle size and drug load of the bortezomib nanoparticles produced as described above.

TABLE B

| Polymer | Load BTZ % | Size (nm) |
|---|---|---|
| 16/5 PLA/PEG | 3 | 107 |
| 30/5 PLA/PEG | 1 | 108 |
| 50/5 PLA/PEG | 2.7 | 106 |
| 65/5 PLA/PEG | 0.2 | 155 |
| 80/5 PLA/PEG | 0.67 | 149 |

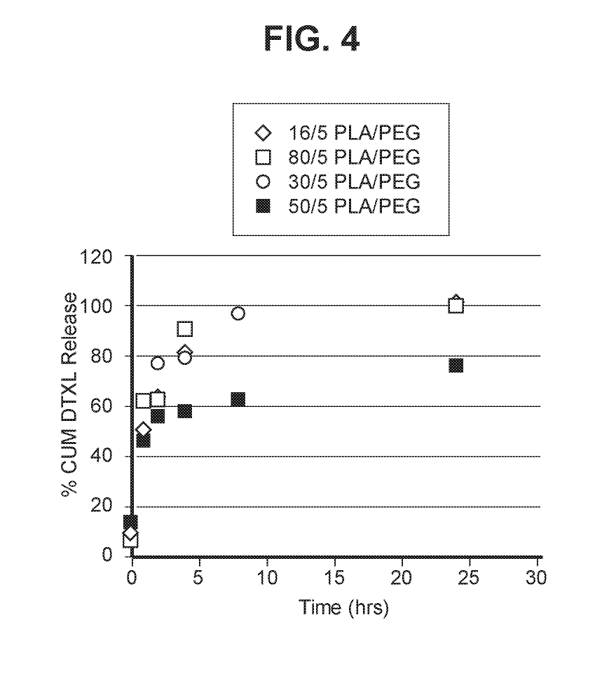

In vitro release test is performed on the above described bortezomib nanoparticles. As depicted in FIG. 4, incorporation of 50-5 PLA-PEG slowed down the release of bortezomib from the nanoparticles.

### Example 4 Vinorelbine Nanoparticles

Vinorelbine nanoparticles comprising either 16-5 or 50-5 PLA-PEG copolymer are prepared using the following formulation: 20% (w/w) theoretical drug and 80% (w/w) polymer-PEG (16/5 or 50-5 PLA-PEG). For nanoparticles comprising 16-5 PLA-PEG: % Total solids=20%; for nanoparticles comprising 50-5 PLA-PEG: % Total solids=30%. For all nanoparticles: solvents used are 21% benzyl alcohol and 79% ethyl acetate (w/w).

Vinorelbine nanoparticles are prepared using a protocol similar to the protocol described above for docetaxel nanoparticles.

Table C provides the particle size and drug load of the vinorelbine nanoparticles produced as described above.

TABLE C

| Polymer | Drug Load % | Size (nm) |
|---|---|---|
| 16/5 PLA/PEG | 10 | 101 |
| 50/5 PLA/PEG | 8.4 | 109 |

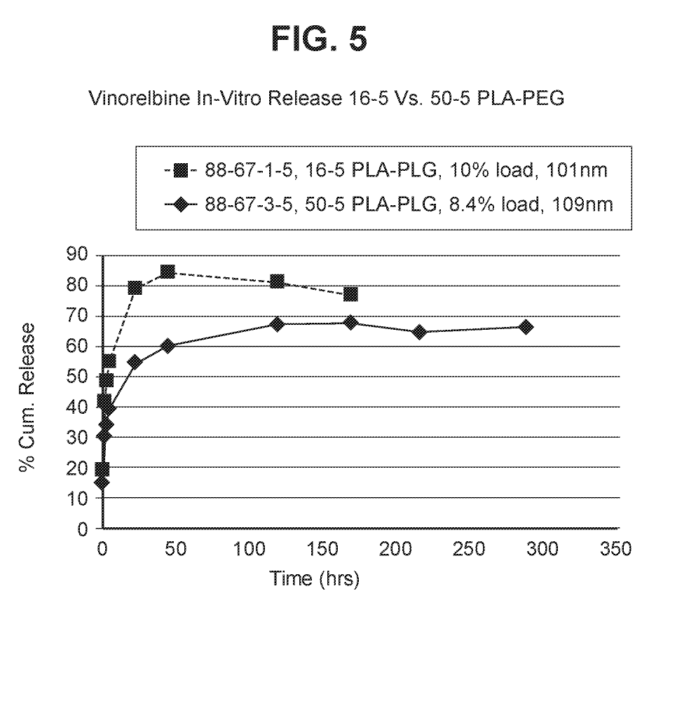

In vitro release test is performed on the above described vinorelbine nanoparticles. As depicted in FIG. 5, incorporation of 50-5 PLA-PEG slowed down the release of vinorelbine from the nanoparticles.

### Example 5 Vincristine Nanoparticles

Vincristine nanoparticles comprising either 16-5 or 50-5 PLA-PEG copolymer are prepared using the following formulation: 20% (w/w) theoretical drug and 80% (w/w) polymer-PEG (16/5 or 50-5 PLA-PEG). For nanoparticles comprising 16-5 PLA-PEG: % Total solids=40%; for nanoparticles comprising 50-5 PLA-PEG: % Total solids=20%. For all nanoparticles: solvents used are 21% benzyl alcohol and 79% ethyl acetate (w/w).

Vincristine nanoparticles are prepared using a protocol similar to the protocol described above for docetaxel nanoparticles.

Table D provides the particle size and drug load of the vinorelbine nanoparticles produced as described above.

TABLE D

| Polymer | Drug Load % | Size (nm) |
|---|---|---|
| 16/5 PLA/PEG | 2.9 | 103 |
| 50/5 PLA/PEG | 2.8 | 122 |

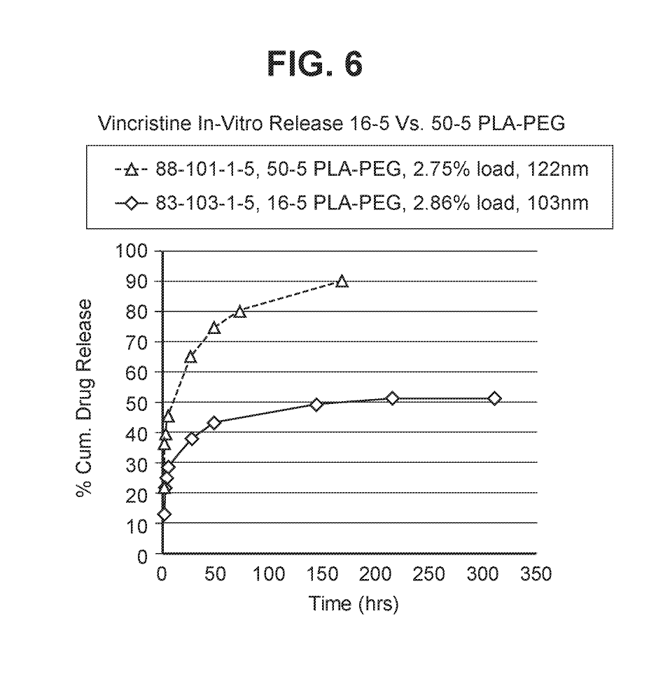

In vitro release test is performed on the above described vincristine nanoparticles. As depicted in FIG. 6, incorporation of 50-5 PLA-PEG slowed down the release of vincristine from the nanoparticles.

### Example 6 Bendamustine Nanoparticles

Bendamustine HCl nanoparticles comprising either 16-5 or 50-5 PLA-PEG copolymer are prepared using the following formulation: 17% (w/w) theoretical drug and 83% (w/w) polymer-PEG (16/5 or 50-5 PLA-PEG) at 20% (w/w) polymer concentration in methylene chloride. Bendamustine HCl is complexed with sodium tetraphenylborate at a 1:1 ratio. % Total solids=40%. Solvents used are 32% benzyl alcohol and 68% methylene chloride (w/w).

Bendamustine nanoparticles are prepared using a protocol similar to the protocol described above for docetaxel nanoparticles.

Table E provides the particle size and drug load of the vinorelbine nanoparticles produced as described above.

TABLE E

| Polymer | Drug Load % | Size (nm) |
|---|---|---|
| 16/5 PLA/PEG | 3.5 | 97 |
| 50/5 PLA/PEG | 2.1 | 202 |

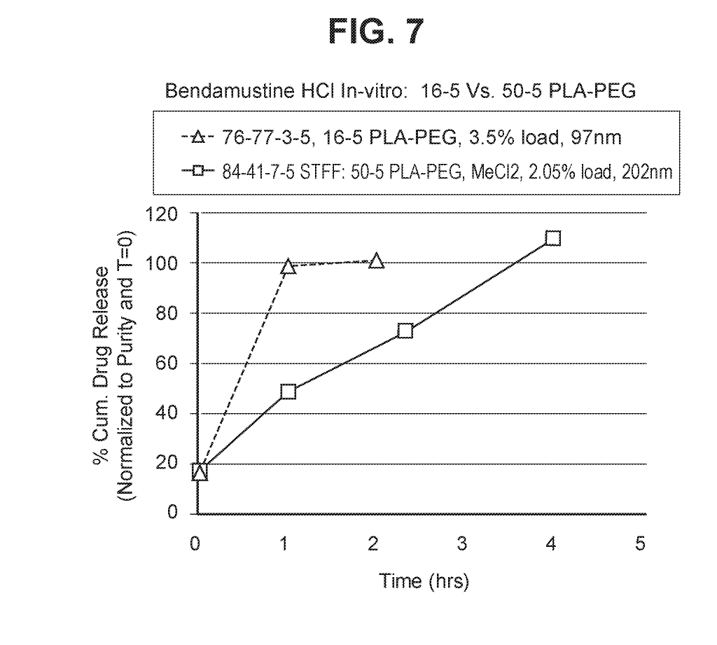

In vitro release test is performed on the above described bendamustine nanoparticles. As depicted in FIG. 7, incorporation of 50-5 PLA-PEG slowed down the release of bendamustine from the nanoparticles.

### Example 7 Diclofenac Nanoparticles

Diclofenac nanoparticles are prepared using a protocol similar to the protocol described above for docetaxel nanoparticles. To determine the in vitro release of diclofenac from the nanoparticles, the nanoparticles were suspended in a release media of 10% Tween 20 in PBS and incubated in a water bath at 37° C. under sink conditions. Samples were collected at specific time points. An ultracentrifugation method was used to separate released drug from the nanoparticles.

TABLE F
Formulation of diclofenac using different molecular weight PLA/PEG
copolymers and homopolymer PLA doping.

| | | Solid | Diclofenac | |
|---|---|---|---|---|
| | API theoretical | concentration | Loading | Size |
| Formulation | load (%) | (%) | (%) | (nm) |
| 16/5 PLA/PEG | 25 | 20 | 9.73 | 98.9 |
| 16/5 PLA/PEG | 20 | 20 | 6.79 | 104.3 |
| 50/5 PLA/PEG | 25 | 20 | 3.41 | 122.3 |
| 50/5 PLA/PEG | 25 | 15 | 5.56 | 92.2 |
| 50/5 PLA/PEG | 25 | 10 | 8.65 | 140.3 |
| 16/5 + 80 kDa PLA | 25 | 20 | 3.29 | 154.5 |

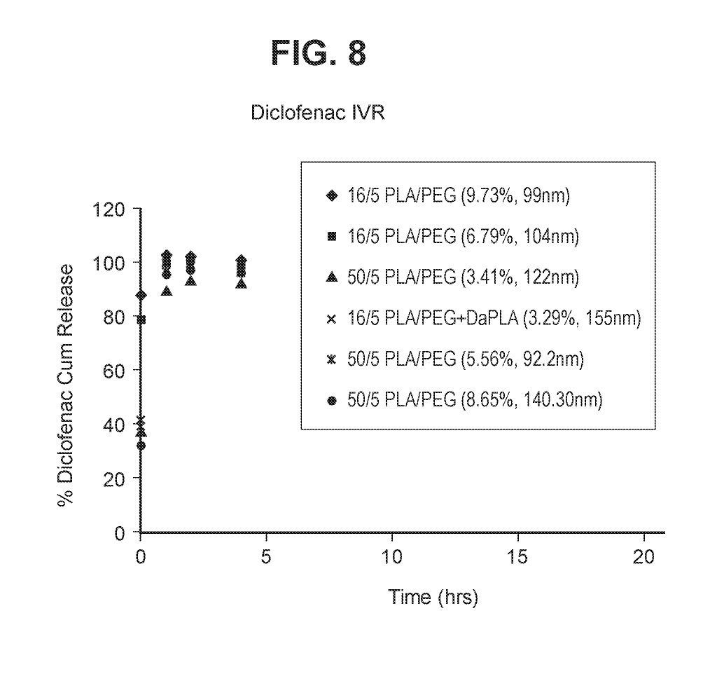

FIG. 8 shows in vitro release of diclofenac from the nanoparticles in Table F. Release of diclofenac was complete within approximately 1-2 hours.

### Example 8 Ketorolac Nanoparticles

Ketorolac nanoparticles are prepared using a protocol similar to the protocol described above for docetaxel nanoparticles.

TABLE G
Formulation of ketorolac using different molecular weight PLA/PEG
copolymers and homopolymer PLA doping.

| | | Solid | Ketorolac | |
|---|---|---|---|---|
| | API theoretical | concentration | Loading | Size |
| Formulation | load (%) | (%) | (%) | (nm) |
| 16/5 PLA/PEG | 30 | 20 | 4.50 | 116.4 |
| 16/5 PLA/PEG | 20 | 30 | 4.86 | 99.8 |
| 50/5 PLA/PEG | 30 | 20 | 0.13 | 109.7 |
| 16/5 | 30 | 20 | 0.17 | 105.6 |
| PLA/PEG + 80 kDa | | | | |
| PLA doped | | | | |

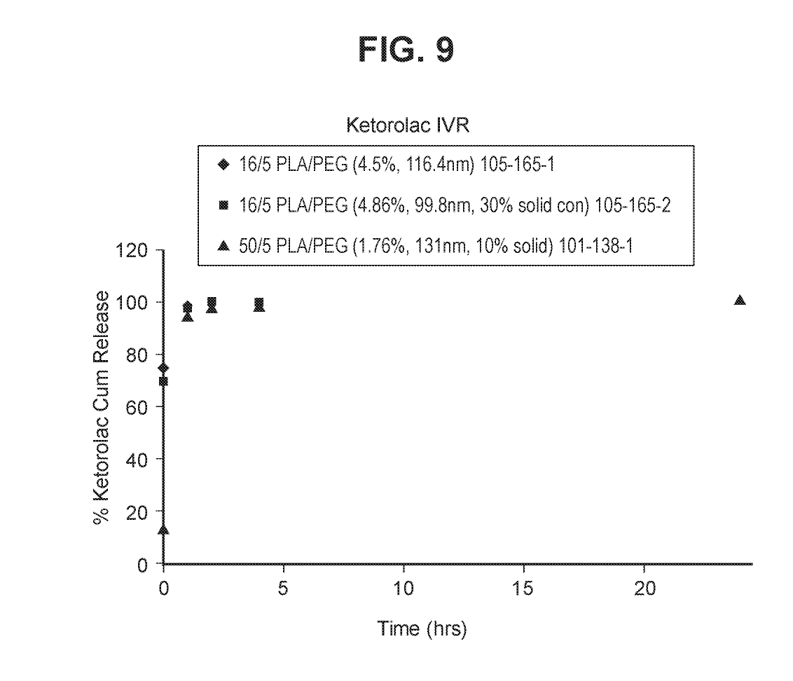

Polymeric nanoparticles made of a copolymer of PLA and PEG were used as carrier in which up to 30% w/w ketorolac (free acid) was entrapped to make the formulation. As can be seen from Table G, the drug loading was found to be about 4.5% for the 16/5 PLA/PEG polymer formulations, indicating 15-24% drug entrapment efficiency. When nanoparticles were formulated with 50/5 PLA/PEG, the entrapment efficiency of ketorolac was 0.13% drug loading and thus, 0.43% encapsulating efficiency. Doping of high molecular weight PLA homopolymer (80 kDa) into 16/5 PLA/PEG also showed 0.17% drug loading. FIG. 9 shows in vitro release of ketorolac from the nanoparticles in Table G. Release of ketorolac was complete within approximately 2 hours.

TABLE H
Impact of solids concentration and sodium cholate (SC) concentration on
ketorolac loading with 50/5 PLA/PEG copolymers.

| | API | Solid | Ketorolac | | % SC & # of |
|---|---|---|---|---|---|
| For- | theoretical | concentration | Loading | Size | homogenizer |
| mulation | load (%) | (%) | (%) | (nm) | passes |
| 50/5 | 30 | 10 | 1.76 | 136.1 | 0.48% |
| PLA/PEG | | | | | 5 passes |
| 50/5 | 30 | 15 | 0.59 | 136.0 | 1.1% |
| PLA/PEG | | | | | 3 passes |
| 50/5 | 30 | 20 | 0.53 | 142.7 | 1.78% |
| PLA/PEG | | | | | 3 passes |

Formulations with solid concentrations of 10%, 15%, and 20% with fixed drug to polymer ratio (30:70) were prepared to investigate solid concentration impact on drug loading (Table H). With decreased solids the level of sodium cholate (SC) was also decreased to achieve appropriate particle size. Formulation with 10% solid concentration with lower SC provided higher drug loading than formulations with 15 and 20% solid.

### Example 9 Rofecoxib Nanoparticles

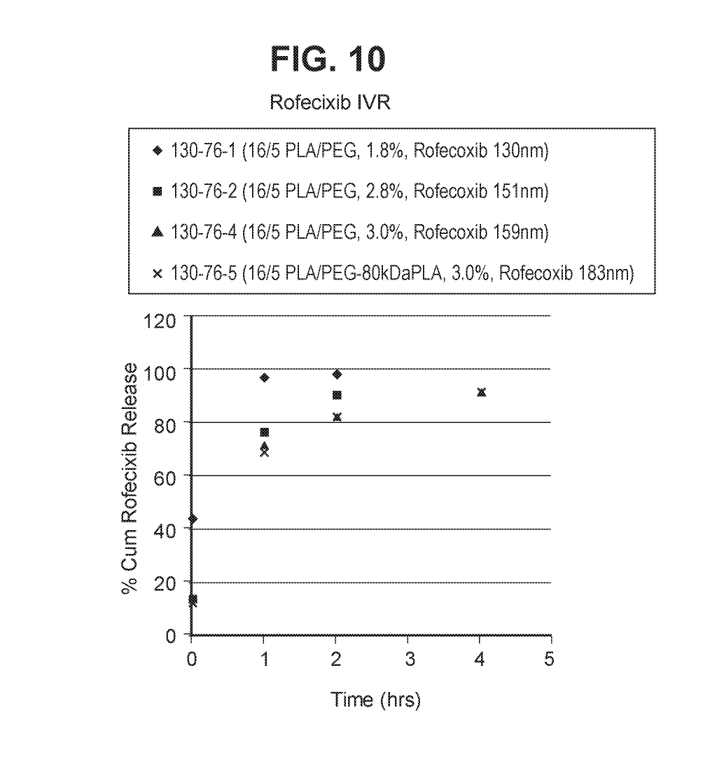

Rofecoxib is encapsulated using above procedures. Table I and FIG. 10 indicate the drug release from nanoparticles made of 16/5 PLA/PEG, 50/5 PLA/PEG, 65/5 PLA/PEG, and 65/5 PLA/PEG with 80 kDa PLA. In vitro release test was performed in the 10% T20 in PBS release medium using centrifuge method

TABLE I
Formulation of Rofecoxib in different molecular weight of PLA/PEG
copolymer and homopolymer PLA doping

| | API | | Rofecoxib | |
|---|---|---|---|---|
| | theorectical | Solid conc | Loading | Size |
| Formulation | load (%) | (%) | (%) | (nm) |
| 16/5 PLA/PEG | 5 | 10% | 1.8 | 130 |
| 50/5 PLA/PEG | 5 | 10% | 2.8 | 151 |
| 65/5 PLA/PEG | 5 | 10% | 3.0 | 159 |
| 65/5 PLA/PEG + | 5 | 10% | 3.0 | 183 |
| 80 kDa PLA | | | | |

### Example 10 Celecoxib Nanoparticles

Celecoxib nanoparticles are encapsulation using above described procedures, with 20%-30% (w/w) theoretical drug, wt. % 70-80% (w/w) Polymer-PEG and/or homopolymers (D,L form), wt. %. % Total Solids=20% and 30% wt. %; Solvents: 21% (BA) benzyl alcohol, 79% (EA) ethyl acetate (w/w), except where noted, (MeCl₂) methylene chloride, wt. % Table J indicates the impact of PLA (polylactic acid) molecular weight and addition of blends of PLA/PLA-PEG on drug load and in vitro release:

TABLE J

| | | | | | % |
|---|---|---|---|---|---|
| | Drug theoretical | | | | release |
| Lot | loading (%) | % Solids | Loading % | size (nm) | T = 1 hr |
| 16k-5k PLA-PEG | 30% | 30% | 15.3 | 122 | 98 |
| 50k-5k PLA-PEG | 30% | 20% | 18.3 | 133 | 96 |
| 65k/5k PLA/PEG | 20% | 20% | 14.49 | 196.3 | 70.9 |
| 16k-5k PLA-PEG/80k PLA lakeshore | 30% | 20% | 15.3 | 134 | 98 |
| (35:35) | | | | | |
| 50k/5k PLA/PEG:80k PLA Lakeshore | 20% | 20% | 12.68 | 189.2 | 88.1 |
| blend (20:60) | | | | | |
| 16k-5k PLA-PEG/50k-5k PLA-PEG | 30% | 20% | 17.6 | 156 | 90 |
| (17.5:52.5) | | | | | |
| 16k/5k PLA-PEG (L-form), BA:MeCl2 | 20% | 20% | 2.58 | 251.3 | 94.9 |
| (21:79 solvent ratio) | | | | | |

The addition of various molecular weight PLA-PEG, blends of 16 k-5 k PLA-PEG, 50 k-5 k PLA-PEG, 80 k PLA to the formulations resulted in drug loads of 13-18%, with in vitro release of 70-98%, drug release after one hour of incubation at 37° C. with orbital shaking under sink conditions.

A formulation produced with L-form 16 k-5 k PLA-PEG (i.e. poly(l-lactic) acid-PEG) made with a solvent blend of benzyl alcohol:methylene chloride (21:79 w/w) ratio resulted in a significantly low drug load of 2.58%, with in vitro release at one hour to be 94.9%. The addition of the L-form of 16 k-5 k PLA-PEG, which is crystalline relative to the D,L-form which is amorphous greatly reduced the encapsulation of drug.

Various drug loaded nanoparticles were prepared, using 5-30% (w/w) theoretical drug, wt. % 70-95% (w/w) Polymer-PEG and/or homopolymers (D,L form), wt. %. % Total Solids=20% and 30% wt. % Solvents: 21% (BA) benzyl alcohol, 79% (EA) ethyl acetate (w/w), wt. %, as shown in Table K

TABLE K
Impact of Celecoxib Drug Load on drug load and in vitro release:

| | Drug | | | | % |
|---|---|---|---|---|---|
| | theoretical | | | size | release |
| | loading (%) | % Solids | Loading % | (nm) | T = 1 hr |
| :50/5 PLA/PEG | 5 | 20% | 3.48 | 146.2 | 79 |
| :16/5 PLA/PEG | 5 | 20% | 2.89 | 128.9 | 99 |
| 75/5 PLA/PEG | 5 | 20% | 4.47 | 223.9 | 44 |
| 16-5 PLA-PEG | 30 | 30% | 15.3 | 122 | 98 |
| 50-5 PLA-PEG | 30 | 20% | 18.3 | 133 | 96 |
| 65/5 PLA/PEG | 20 | 20% | 14.49 | 196.3 | 71 |

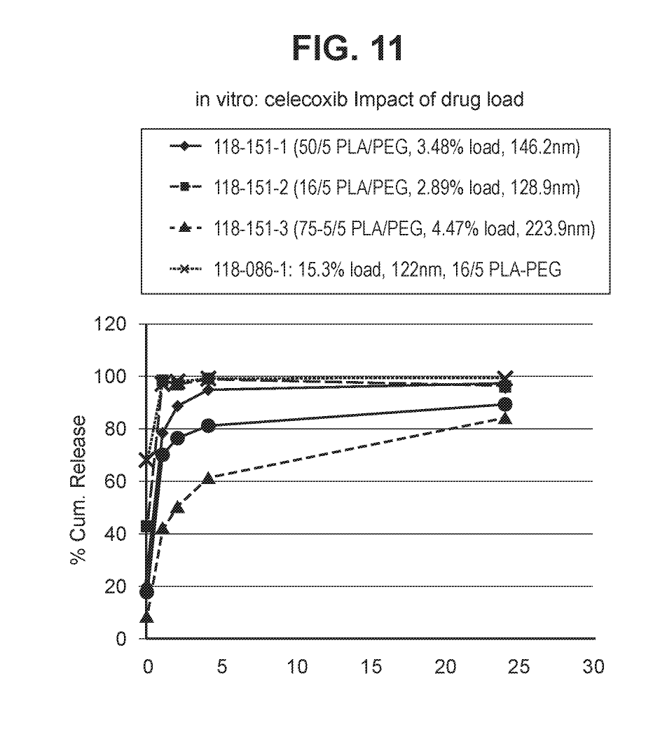

Table K indicates that drug load of the nanoparticles impacts drug release. The 50-5 and 65-5/75-5 PLA-PEG polymer-PEGs were impacted by drug load, while with the 16-5 PLA-PEG, drug load did not impact release. With the 16-5 PLA-PEG polymers, with similar particle size of 122 and 129 nm resulted in 98-99% drug release regardless of drug load. With the 50-5 PLA-PEG polymer, the lower load, 3.48%, resulted in drug release of 79% at the one hour time point while the at the higher load, 18.3%, the drug release was 96%, both at similar particle size. The formulations with 65-5 and 75-5 PLA-PEG, with 14.49% and 4.47% drug load, respectively, and drug release of 71% and 44%, respectively, resulted in the slowest drug release, but with larger particle size of these batches. FIG. 11 shows the complete drug release.

Low drug load nanoparticles were also formed from 5% (w/w) theoretical drug, wt. %; 95% (w/w) Polymer-PEG and/or homopolymers, wt. % Total Solids=20-30%, wt. % Solvents: 21% (BA) benzyl alcohol, 79% (EA) ethyl acetate (w/w), wt. %

TABLE L
Impact of Nanoparticle Particle Size on in
vitro release, at low drug load:

| | Drug | | | | % |
|---|---|---|---|---|---|
| | theoretical | | | size | release |
| | loading (%) | % Solids | Loading % | (nm) | T = 1 hr |
| 50-5 PLA-PEG | 5 | 20% | 4.82 | 310.7 | 28 |
| 50-5 PLA-PEG | 5 | 20% | 4.05 | 195.0 | 61 |
| 50/5 PLA/PEG | 5 | 20% | 3.48 | 146.2 | 79 |
| 16-5 PLA-PEG | 5 | 30% | 3.51 | 164.0 | 96 |
| 16-5 PLA-PEG | 5 | 30% | 4.60 | 370.4 | 76 |

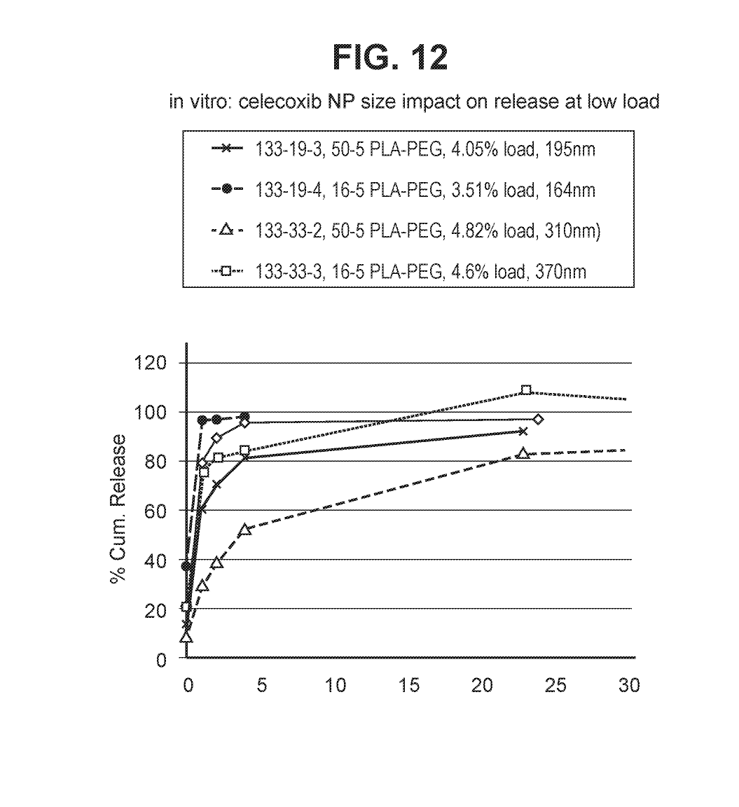

Table L indicates that particle size impacts drug release, as particle size increase in vitro release slows down, at similar drug loads. As particle size increased for the 50-5 PLA-PEG polymer from 146 nm to 310 nm, the drug release at one hour decreased from 79% to 28%. In addition this trend is observed with 16-5 PLA-PEG. With particles of 164 nm the one hour drug release was 96% while with a 370 nm particle the drug release is 76%. FIG. 12, shows the complete release.

### Example 11 Dihexanoate Pt(IV)

Dihexonoate platinum nanoparticles are encapsulation using above described procedures, with benzyl alcohol (drug solubility in BA is 6.1 mg/mL) as polymer and drug solvent, with 5% (w/w) theoretical drug loading; 95% (w/w) Polymer-PEG, 45-5 PLA-PEG; % Total Solids=10%; Solvents: 100% benzyl alcohol. 1 gram batch size: 50 mg of drug; 950 mg of Polymer-PEG: 45-5 PLA-PEG, as shown in Table M, with in-vitro release data shown in table M1

TABLE M
Formulation parameters and nanoparticle properties, using BA
only as organic phase solvent

| | Drug | | | | % SC, | NP |
|---|---|---|---|---|---|---|
| | theoretical | Solid | Loading | size | pass# | Solids |
| Description | loading (%) | conc. | % | (nm) | @psi# | (mg/mL) |
| 45-5 PLA/PEG, | 5 | 10% | 0.23% | 113.5 | 1%, | 6.425 |
| BA only, 1 | | | | | 1@45 | |
| g scale | | | | | psi | |

TABLE M1
In-vitro release

| | Time (hr) | | | | |
|---|---|---|---|---|---|
| | 0 | 1 | 2 | 4 | 24 |
| Cumulative release (%) | 12.0 | 65.6 | 74.3 | 80.2 | 82.20 |

To increase nanoparticle drug loading, a higher theoretical drug loading could be used. Though dihexanoate Pt(IV) has a solubility of 6.1 mg/mL in BA, the highest theoretical drug loading is limited at less than 6%, which may not provided for preparation of higher drug loading nanoparticles.

The solubility of dihexanoate Pt(IV) in DMF was tested to be >112 mg/mL. Compared to BA only, dihexanoate Pt(IV) has much higher solubility when mixing DMF with BA/EA. In a different synthetic study, mixtures of (21/79 BA/EA) and DMF at different ratio were used as organic phase solvent for the purpose of improving theoretical drug loading by enhancing drug solubility. Formulation conditions are as follows: Theoretical drug loading: 10% and 20% (w/w); Polymer-PEG, 45-5 PLA-PEG: 90% and 80% (w/w); % Total Solids: 10%; Solvents: 78% (21/79 benxyl alcohol/ethyl acetate)+22% DMF, and 90% (21/79 benxyl alcohol/ethyl acetate)+10% DMF. 0.5 gram batch size: 50 mg and 100 mg of drug 450 mg and 400 mg of Polymer-PEG, 45-5 PLA-PEG. BA/DMF mixture was used as an organic phase solvent. The preparation is as follows:

Preparation of drug/polymer solution
1.1 To 20 mL glass vial add dihexanoate Pt(IV), 100 mg
1.2 Add 450 mg of dimethylformamide to drug and vortex until it is clear.
1.3 Prepare 21/79 BA/EA mixture by weighing: 21 g of BA and 79 g of EA.
1.4 Add 400 mg of polymer-PEG to a new 20 mL glass vial.
1.5 Add 4050 mg of 21/79 BA/EA mixture to polymer and vortex until it is dissolved.
1.6 Mix drug and polymer solution before formulation by adding polymer solution into drug solution, and vortex.

Preparation of Aqueous Solution: 0.25% sodium cholate, 2% Benzyl Alcohol, 4% Ethyl acetate in Water:
1.7 To 1 L bottle add 2.5 g sodium cholate and 937.5 g of DI water and mix on stir plate until dissolved.
1.8 Add 20 g of benzyl alcohol and 40 g of ethyl acetate to sodium cholate/water and mix on stir plate until dissolved

Formation of emulsion. Ratio of Aqueous phase to Oil phase is 5:1:
1.9 Pour organic phase into aqueous solution and homogenize using hand homogenizer for 10 seconds at room temperature to form course emulsion
1.10 Feed solution through high pressure homogenizer (110S), set pressure to 30 psi on gauge for 1 pass.

Formation of nanoparticles
1.11 Pour emulsion into Quench (D.I. water) at <5 C while stirring on stir plate. Ratio of Quench to Emulsion is 5:1
Add 35% (w/w) Tween 80 in water to quench at ratio of 150:1 Tween 80 to drug.

Concentrate nanoparticles through TFF
1.12 Concentrate quench on TFF with 300 kDa Pall cassette (2 membranes) to ˜200 mL.
1.13 Diafilter ˜30 diavolumes (6 liter) of cold DI water. Bring volume down to minimal volume.
1.14 Add 100 mL of cold water to vessel and pump through membrane to rinse.
1.15 Collect material in glass vial, 50-100 mL

Determination of solids concentration of unfiltered final slurry:
1.16 To tared 20 mL scintillation vial add a volume of final slurry and dry under vacuum at 80° C. in vacuum oven.
1.17 Determine weight of nanoparticles in the volume of slurry dried down
1.18 Add concentrated sucrose (0.111 g/g) to final slurry sample to attain 10% sucrose.

Determination of solids concentration of 0.45 um filtered final slurry:
1.19 Filter about a portion of the final slurry sample before addition of sucrose through 0.45 μm syringe filter
1.20 To tared 20 mL scintillation vial add a volume of filtered sample and dry under vacuum at 80° C. in vacuum oven.

Freeze remaining sample of unfiltered final slurry with sucrose. Table M3 shows the in vitro release of drug from the nanoparticle.

TABLE M3

| | | Drug | | DMF in | | | | |
|---|---|---|---|---|---|---|---|---|
| | | theoretical | | organic | | | % SC, | |
| | | loading | Solid | solvent | Loading | size | pass# @ | NP Solids |
| # | Description | (%) | conc. | (wt %) | % | (nm) | psi# | (mg/mL) |
| 1 | 45-5 PLA/PEG, 3.5/1 | 10 | 10% | 22% | 1.07% | 125.5 | 0.4%, 1@30 psi | 5.125 |
| | (BA/EA)/DMF, 0.5 g | | | | | | | |
| | scale | | | | | | | |
| 2 | 45-5 PLA/PEG, 9/1 | 20 | 10% | 10% | 2.05% | 156.5 | 0.37%, 1@30 psi | 6.25 |
| | (BA/EA)/DMF, 0.5 g | | | | | | | |
| | scale | | | | | | | |
| 3 | 45-5 PLA/PEG, 9/1 | 20 | 10% | 10% | 0.89% | 125.1 | 0.37%, 1@30 psi | 4.75 |
| | (BA/EA)/DMF, 0.5 g | | | | | | | |
| | scale | | | | | | | |
| 4 | 45-5 PLA/PEG, 9/1 | 20 | 10% | 10% | 1.53% | 136.5 | 0.25%, 1@30 psi | 5.15 |
| | (BA/EA)/DMF, 0.5 g | | | | | | | |
| | scale | | | | | | | |

M3. In-vitro release

| | Cumulative release (%) | | | |
|---|---|---|---|---|
| Time (hour) | 1 | 2 | 3 | 4 |
| 0 | 29.8 | 24.0 | 19.3 | 11.5 |
| 1 | 77.7 | 49.9 | 45.0 | 36.7 |
| 2 | 87.2 | 54.0 | 51.2 | 44.2 |
| 4 | 90.4 | 58.8 | 56.2 | 49.6 |
| 24 | 94.7 | 73.7 | — | — |
| 25.8 | — | — | 68.8 | 66.5 |
| 74.3 | — | — | 80.9 | 76.9 |
| 98.3 | — | — | 83.3 | 78.1 |
| 123.1 | — | — | 83.1 | 82.1 |
| 169.6 | — | — | 87.4 | 85.7 |
| 240.3 | — | — | 93.2 | 89.2 |
| 338.3 | — | — | 93.3 | 93.7 |

Dihexanoate Pt(IV) was encapsulated into PLA-PEG nanoparticles through nanoemulsion method. Using BA only as organic phase solvent, a 0.23% drug was loaded, which is relatively low and hard to be improved significantly due to drug's low solubility in BA. A fast in vitro release at 37° C. was observed for this formulation: 65.6% drug was released at 1 hour, and 80.2% drug was release at 4 hour. Using a BA mixture with DMF, formulations with higher theoretical drug loadings are possible. By targeting 10% and 20% drug loading, while keeping 10% solid, four formulations with improved drug loadings were prepared. Lot 1 shows a fast release with 77.7% of drug released at 1 hour, and 90.4% released at 4 hour. When reducing DMF content from 22% to 10%, nanoparticles of lots 2-4 1 all give slower release profiles under the same in vitro conditions. The first hour release is in the range of 33% to 50%, and the 4-hour release is in the range of 50% to 60%, well below 80%. At 24 hour, there still about 30% drug was not released, which was gradually released up to 14-day.

Due to solubility limit in BA and EA, drug loading is relatively low, when using BA only as organic solvent. To further improve drug loading, (21/79 BA/EA) mixture with DMF was successfully adopted as organic solvent of polymer and drug. Nanoparticles with improved drug loading were formulated using nanoemulsion method. Drug release from nanoparticles could be optimized by adjusting the ratio of solvent mixture.

### Example 12 Oxaplatin Nanoparticles

Oxaliplatin solubility in typical nanoemulsion organic solvents, benzyl alcohol, ethyl acetate, and methylene chloride, are all below 250 ug/mL. Oxaliplatin is soluble in water at ˜10 mg/mL. The very low solubility in organic solvent and relatively high solubility in water require different synthetic parameters for encapsulation of oxaliplatin in PLA-PEG nanoparticles through nanoemulsion method; challenges for drug loading with oxaplatin include low theoretical drug loading due to low solubility in organic phase and fast and significant drug leaking during emulsification due to high solubility in water. The following preparation of drug/polymer solution is used:

To 20 mL glass vial add 300 mg oxaliplatin; 1000 mg of dimethylformamide is added to drug and vortex until it is clear. 21/79 BA (benzyl alcohol)/EA (ethyl acetate) mixture is prepared by weighing: 21 g of BA and 79 g of EA.

700 mg of polymer-PEG is added to a new 20 mL glass vial. 3000 mg of 21/79 BA/EA mixture is added to polymer and vortex until it is dissolved. The drug and polymer solution are mixed before formulation by adding polymer solution into drug solution, and vortex.

An aqueous solution of 1% sodium cholate, 45% Tetrahydrofuran in Water is prepared using a 500 mL bottle and adding 5 g sodium cholate and 270 g of DI water and mix on stir plate until dissolved. 225 g of tetrahydrofuran is added to sodium cholate/water and mix on stir plate until dissolved.

An emulsion is formed with a ratio of Aqueous phase to Oil phase of 5:1: the organic phase is poured into aqueous solution and homogenize using hand homogenizer for 10 seconds at room temperature to form course emulsion. The solution is fed through high pressure homogenizer (110S), set pressure to 20 psi on gauge for 1 pass. The nanoparticles are formed by pouring the emulsion into quench (D.I. water) at <5 C while stirring on stir plate. The ratio of quench to emulsion is 5:1. The nanoparticles are concentrated through TFF by concentrating the quench on TFF with 300 kDa Pall cassette (2 membranes) to ˜200 mL, and Diafilter ˜20 diavolumes (4 liter) of cold DI water. The volume is brought down to minimal volume; 100 mL of cold water is added to the vessel and pump through membrane to rinse; and the material in glass vial is gathered: 50-100 mL. The determination of solids concentration of unfiltered final slurry is obtained by adding a volume of final slurry to a tared 20 mL scintillation vial and dry under vacuum at 80° C. in vacuum oven. The weight of nanoparticles is determined in the volume of slurry dried down and concentrated sucrose (0.111 g/g) is added to the final slurry sample to attain 10% sucrose.

Solids concentration of 0.45 um filtered final slurry was determined by filtering about a portion of the final slurry sample before addition of sucrose through 0.45 μm syringe filter; a volume of filtered sample was added to tared 20 mL scintillation vial and dried under vacuum at 80° C. in vacuum oven. The remaining sample of unfiltered final slurry was frozen with sucrose.

To improve drug solubility in organic phase and use higher theoretical drug loading, mixtures of BA with DMSO or (21/79 BA/EA) with DMSO were used as organic solvent. Because oxaliplatin is very soluble in DMSO, above 500 mg/mL, the mixture of BA or (21/79 BA/EA) with DMSO could dissolve much more oxaliplatin than BA or 21/79 BA/EA only. In addition, aqueous phase composition was modified by adding 45% THF. Oxaliplatin solubility in 1/1 water/THF mixture is ˜1 mg/mL, which is much lower than its solubility in water, ˜10 mg/mL. The formulation was prepared using modified organic and aqueous phases: 30% (w/w) theoretical drug loading; 70% (w/w) Polymer-PEG, 45-5 PLA-PEG; % Total Solids=20%; Solvent: mixture of 75% benzyl alcohol and 25% DMSO. 1 gram batch size: 300 mg of drug; 700 mg of Polymer-PEG: 45-5 PLA-PEG

TABLE N
Formulation parameters and nanoparticle properties

| | Drug | | | | | % | NP |
|---|---|---|---|---|---|---|---|
| | theo- | | | | | SC, | Sol- |
| | retical | Sol- | DMF | | | pass# | ids |
| | loading | id | (wt | Loading | size | @ | (mg/ |
| Description | (%) | conc | %) | % | (nm) | psi# | mL) |
| 50-5 | 30 | 20% | 25 | 0.19% | 141.7 | 1%, | 5.625 |
| PLA-PEG, 1/1 | | | | | | 1@20 | |
| THF/water | | | | | | psi | |
| with 1% SC, | | | | | | | |
| 3/1 (BA/EA)/ | | | | | | | |
| DMSO | | | | | | | |

By improving solubility of oxaliplatin in organic phase and suppressing solubility in aqueous phase, oxaliplatin was encapsulated into PLA-PEG nanoparticles through nanoemulsion method under modified conditions: mixture of BA/EA with DMSO as organic phase solvent, and mixture of water with THF as aqueous phase solvent. Under these conditions worked useful particle size and solid concentration were obtained for final product.

### Example 13 Nanoparticles with Therapeutic Agents

The following nanoparticles shown in Table O are prepared using the above outlined protocols:

TABLE O

| | | Weight percent | |
|---|---|---|---|
| | | therapeutic | Weight percent |
| Therapeutic Agent | Polymer | agent | polymer |
| Diclofenac | 30:5 PLA-PEG or | Particles A. 5% | Particles A. 95% |
| | 30:5PLGA-PEG | B. 4% | B. 96% |
| | | C. 7% | C. 97% |
| | | D. 10% | D 90% |
| | | E. 12% | E. 88% |
| | | F. 15% | F 85% |
| Diclofenac | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Therapeutic Agent | Polymer | Weight percent agent | Weight percent polymer |
| Oxaplatin | 30:5 PLA-PEG or | A. 5 | A. 95 |
| | 30:5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Diclofenac | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Oxaplatin | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 30:5 PLA-PEG or | A. 5 | A. 95 |
| | 30:5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Cisplatin | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 30:5 PLA-PEG or | A. 5 | A. 95 |
| | 30:5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Dihexanoate Pt | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |
| vinorelbine | 30:5 PLA-PEG or | A. 5 | A. 95 |
| | 30:5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Vinorelbine | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 30:5 PLA-PEG or | A. 5 | A. 95 |
| | 30:5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Vincristine | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 30:5 PLA-PEG or | A. 5 | A. 95 |
| | 30:5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Bendamustine | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 30:5 PLA-PEG or | A. 5 | A. 95 |
| | 30:5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Ketorolac | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 30:5 PLA-PEG or | A. 5 | A. 95 |
| | 30:5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Rofecoxib | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 30:5 PLA-PEG or | A. 5 | A. 95 |
| | 30:5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 50:5 PLA-PEG or | A. 5 | A. 95 |
| | 50:5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 80:5 PLA-PEG or | A. 5 | A. 95 |
| | 80:5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 75:5 PLA-PEG or | A. 5 | A. 95 |
| | 75:5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 47:5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 45:5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 65:5 PLA-PEG or | A. 5 | A. 95 |
| | 65:5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 30:10 PLA-PEG or | A. 5 | A. 95 |
| | 30:10PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 50:10 PLA-PEG or 50:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 80:10 PLA-PEG or | A. 5 | A. 95 |
| | 80:10PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 75:10 PLA-PEG or | A. 5 | A. 95 |
| | 75:10PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 47:10 PLA-PEG or | A. 5 | A. 95 |
| | 47:10PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 45:10 PLA-PEG or 45:10 | A. 5 | A. 95 |
| | PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 65:10 PLA-PEG or | A. 5 | A. 95 |
| | 65:10PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 30:7.5 PLA-PEG or | A. 5 | A. 95 |
| | 30:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 50:7.5 PLA-PEG or | A. 5 | A. 95 |
| | 50:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 80:7.5 PLA-PEG or | A. 5 | A. 95 |
| | 80:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 75:7.5 PLA-PEG or | A. 5 | A. 95 |
| | 75:7.5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 47:7.5 PLA-PEG or | A. 5 | A. 95 |
| | 47:5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 45:7.5 PLA-PEG or | A. 5 | A. 95 |
| | 45:5PLGA-PEG | B. 4 | B. 96 |
| | | C. 7 | C. 97 |
| | | D. 10 | D 90 |
| | | E. 12 | E. 88% |
| | | F. 15 | F 85% |
| Doxetaxel | 65:7.5 PLA-PEG or | A. 5 | A. 95 |
| | 65:7.5PLGA-PEG | B. 4 | B. 96 |
| | | C. 7 | C. 97 |
| | | D. 10 | D 90 |
| | | E. 12 | E. 88% |
| | | F. 15 | F 85% |

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, web sites, and other references cited herein are hereby expressly incorporated herein in their entireties by reference.

1. A biocompatible, therapeutic polymeric nanoparticle comprising:
about 0.1 to about 40 weight percent of a therapeutic agent; and
about 10 to about 95 weight percent biocompatible polymer, wherein the biocompatible polymer is selected from the group consisting of:
a) a diblock poly(lactic) acid-poly(ethylene)glycol copolymer, wherein the diblock poly(lactic) acid-poly(ethylene)glycol copolymer comprises poly(lactic) acid having a number average molecule weight of about 30 kDa to about 90 kDa; and
b) a diblock poly(lactic)-co-poly(glycolic) acid-poly(ethylene)glycol copolymer, wherein the diblock poly(lactic)-co-poly(glycolic) acid-poly(ethylene)glycol copolymer comprises poly(lactic)-co-poly(glycolic) acid having a number average molecule weight of about 30 kDa to about 90 kDa.

2. The biocompatible, therapeutic polymeric nanoparticle of claim 1, wherein the biocompatible polymer is selected from the group consisting of:
a) a diblock poly(lactic) acid-poly(ethylene)glycol copolymer, wherein the diblock poly(lactic) acid-poly(ethylene)glycol copolymer comprises poly(lactic) acid having a number average molecule weight of about 40 kDa to about 90 kDa; and
b) a diblock poly(lactic)-co-poly(glycolic) acid-poly(ethylene)glycol copolymer, wherein the diblock poly(lactic)-co-poly(glycolic) acid-poly(ethylene)glycol copolymer comprises poly(lactic)-co-poly(glycolic) acid having a number average molecule weight of about 40 kDa to about 90 kDa.

3. The biocompatible, therapeutic polymeric nanoparticle of claim 1, wherein the diblock poly(lactic) acid-poly(ethylene)glycol copolymer or the diblock poly(lactic)-co-poly(glycolic) acid-poly(ethylene)glycol copolymer comprises poly(ethylene) glycol having a molecular weight of about 4 kDa to about 6 kDa.

4. The biocompatible, therapeutic polymeric nanoparticle of claim 1, wherein said diblock poly(lactic) acid-poly(ethylene)glycol copolymer comprises poly(lactic) acid having a number average molecular weight of about 50 kDa to about 80 kDa and poly(ethylene)glycol having a number average molecular weight of about 5 kDa.

5. The biocompatible, therapeutic polymeric nanoparticle of claim 1, wherein said diblock poly(lactic) acid-poly(ethylene)glycol copolymer comprises poly(lactic) acid having a number average molecular weight of about 40 kDa to about 60 kDa and poly(ethylene)glycol having a number average molecular weight of about 5 kDa.

6. The biocompatible, therapeutic polymeric nanoparticle of claim 1, wherein said diblock poly(lactic) acid-poly(ethylene)glycol copolymer comprises poly(lactic) acid having a number average molecular weight of about 50 kDa and poly(ethylene)glycol having a number average molecular weight of about 5 kDa.

7. The biocompatible, therapeutic polymeric nanoparticle of claim 1, wherein said diblock poly(lactic) acid-poly(ethylene)glycol copolymer comprises poly(lactic) acid having a number average molecular weight of about 45 kDa and poly(ethylene)glycol having a number average molecular weight of about 5 kDa.

8. The biocompatible, therapeutic polymeric nanoparticle of claim 1, wherein said diblock poly(lactic) acid-poly(ethylene)glycol copolymer comprises poly(lactic) acid having a number average molecular weight of about 30 kDa and poly(ethylene)glycol having a number average molecular weight of about 5 kDa.

9. The biocompatible, therapeutic polymeric nanoparticle of claim 1, wherein the therapeutic agent is selected from the group consisting of vinca alkaloids, platinum chemotherapeutic agents, nitrogen mustard agents, taxanes, mTOR inhibitors, non-steroidal anti-inflammatory drugs, boronate esters or peptide boronic acid compounds, and epothilone.

10. The biocompatible, therapeutic polymeric nanoparticle of claim 1, wherein the therapeutic agent is selected from the group consisting of vinblastine, vinorelbine, vindesine, vincristine; docetaxel, sirolimus, temsirolimus, everolimus, bortezomib, cisplatin, oxaplatin, dihexanoate Pt, and epothilone.

11. The biocompatible, therapeutic polymeric nanoparticle of claim 1, wherein the therapeutic agent is docetaxel.

12. The biocompatible, therapeutic polymeric nanoparticle of claim 1, wherein the therapeutic agent is selected from the group consisting of ketorolac, diclofenac, rofecoxib, and celecoxib.

13. The biocompatible, therapeutic polymeric nanoparticle of claim 1, comprising about 1 to about 20 weight percent therapeutic agent, and about 50 to about 90 weight percent diblock copolymer.

14. The biocompatible, therapeutic polymeric nanoparticle of claim 1, comprising about 2 to about 20 weight percent therapeutic agent.

15. The biocompatible, therapeutic polymeric nanoparticle of claim 1, comprising about 3 to about 6 weight percent therapeutic agent.

16. The biocompatible, therapeutic polymeric nanoparticle of claim 1, comprising about 4 to about 10 weight percent therapeutic agent.

17. The biocompatible, therapeutic polymeric nanoparticle of claim 1, comprising about 6 to about 10 weight percent therapeutic agent.

18. The biocompatible, therapeutic polymeric nanoparticle of claim 1, further comprising poly(lactic) acid or poly(lactic)-co-poly(glycolic) acid.

19. The biocompatible, therapeutic polymeric nanoparticle of claim 14, wherein the poly(lactic) acid has a number average molecular weight of about 50 kDa to about 100 kDa.

20. A pharmaceutical composition comprising a plurality of biocompatible, therapeutic polymeric nanoparticles of claim 1, and a pharmaceutically acceptable excipient.

21-34. (canceled)
